(19) United States
(12) Patent Application Publication
(10) Pub. No.: US 2023/0095276 A1
(43) Pub. Date: Mar. 30, 2023

(54) ESTERS OF A RETINOID AND A TOCOPHEROL OR TERT-BUTYLHYDROQUINONE AND PREPARATIONS THEREOF

(71) Applicant: Conagen Inc., Bedford, MA (US)

(72) Inventors: Suizhou Yang, North Chelmsford, MA (US); Dechuan Meng, Braintree, MA (US); Yisheng Wu, Bedford, MA (US)

(73) Assignee: Conagen Inc., Bedford, MA (US)

(21) Appl. No.: 17/889,047

(22) Filed: Aug. 16, 2022

Related U.S. Application Data
(60) Provisional application No. 63/234,233, filed on Aug. 17, 2021.

(51) Int. Cl. A61K 8/49; A61Q 19/08; C07D 311/72

(52) U.S. Cl. CPC A61K 8/498; A61Q 19/08; C07D 311/72

(57) ABSTRACT

The present disclosure provides esters of a retinoid and an alcohol selected from the group consisting of tocopherols and tert-butylhydroquinone, such as esters of Formula (I). The present disclosure also provides compositions and kits comprising the esters, methods of producing the esters, and methods of using the esters (e.g., for treating or preventing a skin disease, slowing the ageing of the skin, improving the appearance of the skin, or modulating a retinoid receptor).

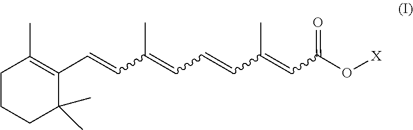

### RELATED APPLICATIONS

The present application claims priority to U.S. Provisional Patent Application No. 63/234,233, filed Aug. 17, 2021, which is incorporated herein by reference.

### BACKGROUND OF THE DISCLOSURE

Certain retinol derivatives (e.g., retinol, retinyl esters, and retinoic esters) are useful as ingredients in skin-care products. International PCT Application Publication No. WO 1991001301 discloses compounds and methods for treating skin for acne or psoriasis. Mukherjee et al., Clinical Interventions in Aging, 2006, 1, 327-348 discloses compounds in the treatment of skin aging. Xiang et al., Hunan Daxue Xuebao, Ziran Kexueban, 2004, 31, 6-10 discloses a synthesis of certain isotretinoin derivatives. Vitamin E has been used in skin-care products. There is a need for improved skin-care products.

### SUMMARY OF THE DISCLOSURE

In one aspect, the present disclosure provides an ester of Formula (I):

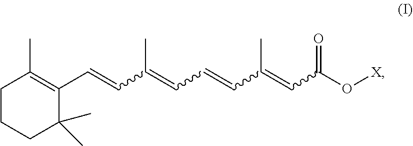

or a tautomer, isotopically labeled compound, solvate, polymorph, or co-crystal thereof, wherein X is of the formula:

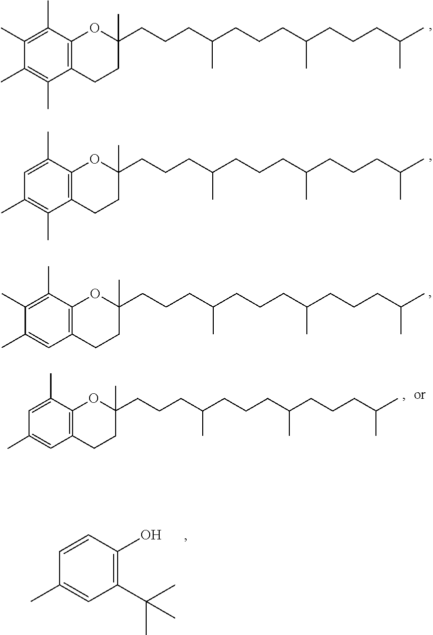

In certain embodiments, the ester is of the formula:

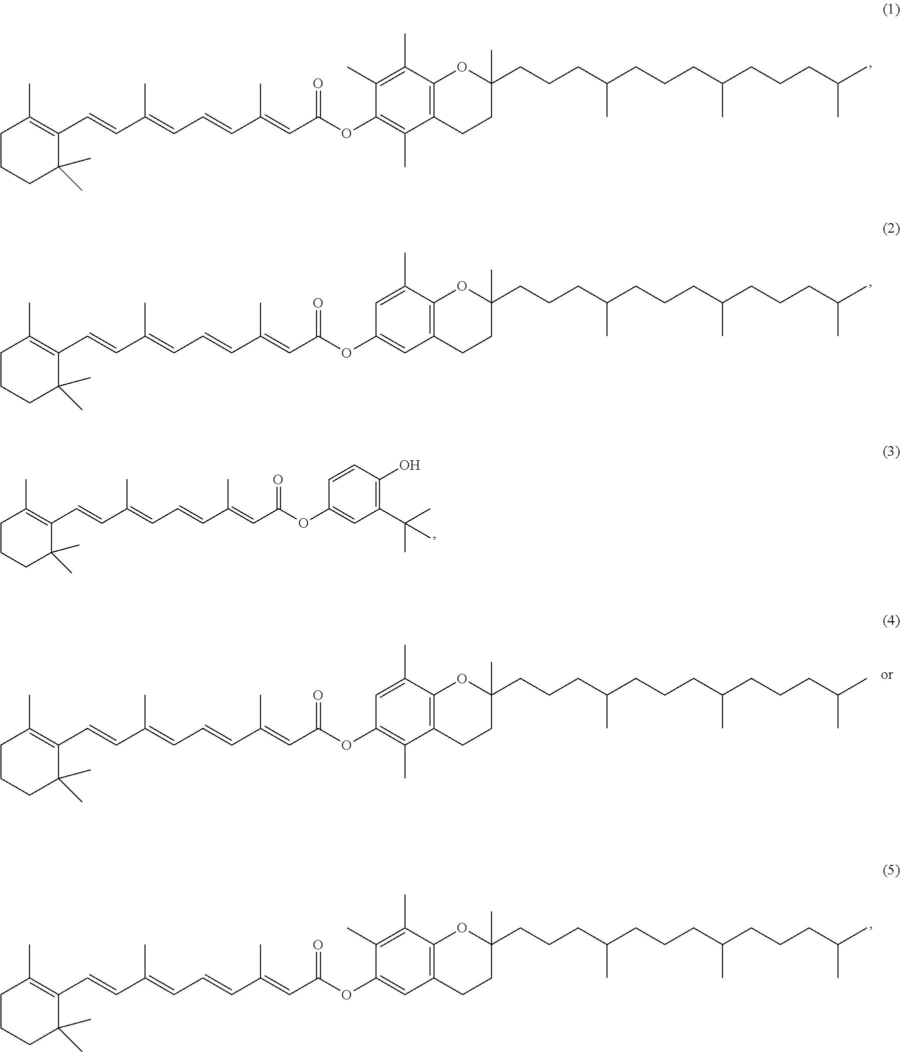 [SMILES: CC1=C(/C=C/C(C)=C/C=C/C(C)=C/C(=O)Oc2c(C)c(C)c3c(c2C)CCC(C)(CCCC(C)CCCC(C)CCCC(C)C)O3)C(C)(C)CCC1 ; CC1=C(/C=C/C(C)=C/C=C/C(C)=C/C(=O)Oc2cc(C)c3c(c2)CCC(C)(CCCC(C)CCCC(C)CCCC(C)C)O3)C(C)(C)CCC1 ; CC1=C(/C=C/C(C)=C/C=C/C(C)=C/C(=O)Oc2ccc(O)c(C(C)(C)C)c2)C(C)(C)CCC1 ; CC1=C(/C=C/C(C)=C/C=C/C(C)=C/C(=O)Oc2cc(C)c3c(c2C)CCC(C)(CCCC(C)CCCC(C)CCCC(C)C)O3)C(C)(C)CCC1 ; CC1=C(/C=C/C(C)=C/C=C/C(C)=C/C(=O)Oc2cc3c(c(C)c2C)OC(C)(CCCC(C)CCCC(C)CCCC(C)C)CC3)C(C)(C)CCC1]

or a tautomer, isotopically labeled compound, solvate, polymorph, or co-crystal thereof.

The esters, and the tautomers, isotopically labeled compounds, solvates, polymorphs, and co-crystals thereof, may be formed from a retinoid and an alcohol selected from the group consisting of α-tocopherol (e.g., (±)α-tocopherol), β-tocopherol (e.g., rac-ρ-tocopherol), γ-tocopherol (e.g., (±)γ-tocopherol, (+)γ-tocopherol), δ-tocopherol (e.g., (+)δ-tocopherol), and tert-butylhydroquinone (TBHQ). The esters may be useful in improving skin health and/or reducing skin ageing. The esters may be advantageous over the retinoids and alcohols that form the esters. The advantages may be at least in part due to that the esters comprise both a retinoid moiety and an alcohol moiety.

In another aspect, the present disclosure provides a method of producing the ester, or a tautomer, isotopically labeled compound, solvate, polymorph, or co-crystal thereof, the method comprising incubating a second reaction mixture for a second time duration sufficient to produce the ester, tautomer, isotopically labeled compound, solvate, polymorph, or co-crystal, wherein the second reaction mixture comprises:

(a) an anhydride of Formula (B):

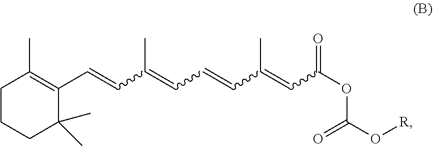

or a tautomer, isotopically labeled compound, solvate, polymorph, or co-crystal thereof, wherein R is substituted or unsubstituted alkyl;

(b) an alcohol of Formula (C):

HO—X (C),

or a tautomer, isotopically labeled compound, solvate, polymorph, or co-crystal thereof;

(c) a second solvent;

(d) optionally a second base; and

(e) optionally an esterification catalyst.

In certain embodiments, the method further comprises incubating a third reaction mixture comprising the second reaction mixture and ethanolamine, or a salt thereof, for a third time duration.

In certain embodiments, the method further comprises incubating a first reaction mixture for a first time duration sufficient to produce (a) of the second reaction mixture, wherein:

the first reaction mixture comprises:
(a) an acid of Formula (A1):

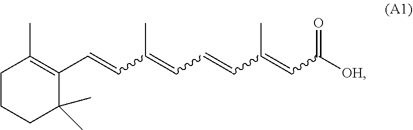 [SMILES: CC(C=CC1=C(C)CCCC1(C)C)=CC=CC(C)=CC(=O)O]

or a tautomer, isotopically labeled compound, salt, solvate, polymorph, or co-crystal thereof;
(b) a chloroformate of Formula (A2):

Cl—C(═O)—O—R (A2),

or an isotopically labeled compound, solvate, polymorph, or co-crystal thereof, wherein R is substituted or unsubstituted alkyl;
(c) a first solvent; and
(d) optionally a first base; and

the step of incubating a first reaction mixture is prior to the step of incubating the second reaction mixture.

In certain embodiments, the method of producing the ester, or a tautomer, isotopically labeled compound, solvate, polymorph, or co-crystal thereof, comprises the steps shown in the scheme:

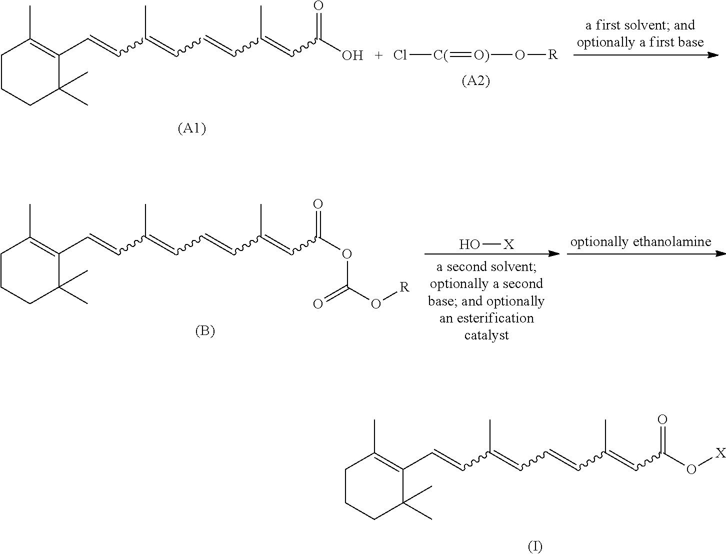

In another aspect, the present disclosure provides a composition comprising:

the ester, or a tautomer, isotopically labeled compound, solvate, polymorph, or co-crystal thereof; and

optionally an excipient.

In another aspect, the present disclosure provides a composition comprising the ester, or a tautomer, isotopically labeled compound, solvate, polymorph, or co-crystal thereof, produced by the method.

In another aspect, the present disclosure provides a kit comprising:

the ester, or a tautomer, isotopically labeled compound, solvate, polymorph, or co-crystal thereof, or the composition; and

optionally instructions for using the ester, tautomer, isotopically labeled compound, solvate, polymorph, co-crystal, or composition.

In another aspect, the present disclosure provides a method of treating a skin disease in a subject in need thereof comprising administering to the subject in need thereof an effective amount of the ester, or a tautomer, isotopically labeled compound, solvate, polymorph, or co-crystal thereof, or the composition.

In another aspect, the present disclosure provides a method of preventing a skin disease in a subject in need thereof comprising administering to the subject in need thereof an effective amount of the ester, or a tautomer, isotopically labeled compound, solvate, polymorph, or co-crystal thereof, or the composition.

In another aspect, the present disclosure provides a method of slowing the ageing of the skin or improving the appearance of the skin of a subject comprising administering to the subject an effective amount of the ester, or a tautomer, isotopically labeled compound, solvate, polymorph, or co-crystal thereof, or the composition.

In another aspect, the present disclosure provides a method of modulating a retinoid receptor in a subject, biological sample, tissue, or cell comprising administering to the subject or contacting the biological sample, tissue, or cell with an effective amount of the ester, or a tautomer, isotopically labeled compound, solvate, polymorph, or co-crystal thereof, or the composition.

While the disclosure is susceptible to various modifications and alternative forms, specific embodiments thereof are described below. It should be understood, however, that the detailed description presented herein are not intended to limit the disclosure to the particular embodiments disclosed, but on the contrary, the intention is to cover all modifications, equivalents, and alternatives falling within the spirit and scope of the disclosure as defined by the appended claims.

Other features and advantages of this disclosure will become apparent in the following detailed description of certain embodiments of the disclosure.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds (e.g., esters) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

In a formula, the bond [symbol] is a single bond where the stereochemistry is not specified.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C_{4_5}, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ═CH). The moieties ═CH₂ and ═CH are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃,

—CF₂CF₃, —CF₂CF₂CF₃, —CCl₃, —CFCl₂, —CF₂Cl, and the like.

“Alkenyl” refers to a radical of a straight-chain or branched hydrocarbon group having from to 20 carbon atoms, one or more (e.g., two, three, or four, as valency permits) carbon-carbon double bonds, and no triple bonds (“C₂₋₂₀ alkenyl”). In some embodiments, an alkenyl group has 2 to carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

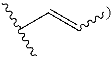

may be in the (E)- or (Z)-configuration.

“Alkynyl” refers to a radical of a straight-chain or branched hydrocarbon group having from to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C≡C triple bonds in the carbocyclic ring is referred to as “cycloalkynyl.” “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. In certain embodiments, the carbocyclyl includes oxo substituted thereon.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-13 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continues to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic. In certain embodiments, the heterocyclyl includes oxo substituted thereon.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include aziridinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In certain embodiments, the heteroaryl group is 5-6 membered, monocyclic. In certain embodiments, the heteroaryl group is 8-14 membered, bicyclic.

Exemplary 5-membered heteroaryl groups containing one heteroatom include pyrrolyl, furanyl, and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R′)₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases.

Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The provided compounds may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro- forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “isotopically labeled compound” refers to a derivative of a compound that only structurally differs from the compound in that at least one atom of the derivative includes at least one isotope enriched above (e.g., enriched between 3- and 10-fold, between 10- and 30-fold, between 30- and 100-fold, between 100- and 300-fold, between 300- and 1,000-fold, between 1,000- and 3,000-fold, or between 3,000- and 10,000-fold above) its natural abundance, whereas each atom of the compound includes isotopes at their natural abundances. In certain embodiments, the isotope enriched above its natural abundance is ²H. In certain embodiments, only one, two, three, four, or five hydrogen atoms of the isotopically labeled compound include ²H above its natural abundance. In certain embodiments, the isotope enriched above its natural abundance is ¹³C or ¹⁸O. In certain embodiments, only one, two, or three carbon atoms of the isotopically labeled compound include ¹³C above its natural abundance. In certain embodiments, only one or two oxygen atoms of the isotopically labeled compound include ¹⁸O above its natural abundance.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a provided compound and an acid), wherein each of the components is independently an atom, ion, or molecule. In certain embodiments, none of the components is a solvent. In certain embodiments, at least one of the components is a solvent. A co-crystal of a provided compound and an acid is different from a salt formed from a provided compound and the acid. In the salt, a provided compound is complexed with the acid in a way that proton transfer (e.g., a complete proton transfer) from the acid to a provided compound easily occurs at room temperature. In the co-crystal, however, a provided compound is complexed with the acid in a way that proton transfer from the acid to a provided herein does not easily occur at room temperature. In certain embodiments, in the co-crystal, there is no proton transfer from the acid to a provided compound. In certain embodiments, in the co-crystal, there is partial proton transfer from the acid to a provided compound. Co-crystals may be useful to improve the properties (e.g., solubility, stability, and ease of formulation) of a provided compound.

The solid forms described herein (e.g., tautomers, stereoisomers, isotopically labeled compounds, salts, solvates, polymorphs, and co-crystals) include all combinations thereof. For example, a tautomer, isotopically labeled compound, salt, solvate, polymorph, or co-crystal of a compound described herein includes, e.g., a polymorph of a solvate of a salt of an isotopically labeled compound of a tautomer of the compound described herein. In certain embodiments, the solid form is cosmetically acceptable. In certain embodiments, the solid form is pharmaceutically acceptable.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the subject is a mammal. The subject may be a male or female and at any stage of development. A non-human animal may be a transgenic animal.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “composition” and “formulation” are used interchangeably.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence. The treatment may be therapeutic treatment (not including prevention or prophylactic treatment).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

An “effective amount” of a compound refers to an amount sufficient to elicit the desired biological response. An effective amount of a compound may vary depending on such factors as the desired biological endpoint, the pharmacokinetics of the compound, the condition being treated, the mode of administration, and the age and health of the subject. In certain embodiments, the effective amount is a therapeutically effective amount. In certain embodiments, the effective amount is a prophylactically effective amount. In certain embodiments, the effective amount is the amount of the compound in a single dose. In certain embodiments, the effective amount is the combined amounts of the compound in multiple doses.

A “therapeutically effective amount” of a compound is an amount sufficient to provide a therapeutic benefit in the treatment of a condition or to delay or minimize one or more signs and/or symptoms associated with the condition. In certain embodiments, the therapeutically effective amount is an amount that improves overall therapy, reduces or avoids symptoms, signs, or causes of the condition, and/or enhances the therapeutic efficacy of another therapeutic agent.

A “prophylactically effective amount” of a compound is an amount sufficient to prevent a condition, or one or more signs and/or symptoms associated with the condition or prevent its recurrence. In certain embodiments, the prophylactically effective amount is an amount that improves overall prophylaxis and/or enhances the prophylactic efficacy of another prophylactic agent.

### DETAILED DESCRIPTION OF CERTAIN EMBODIMENTS OF THE DISCLOSURE

### Esters and Method of Producing the Esters

In one aspect, the present disclosure provides an ester of Formula (I):

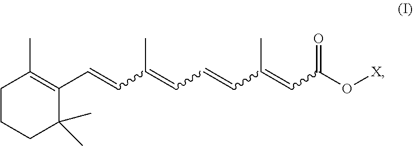

or a tautomer, isotopically labeled compound, solvate, polymorph, or co-crystal thereof, wherein X is of the formula:

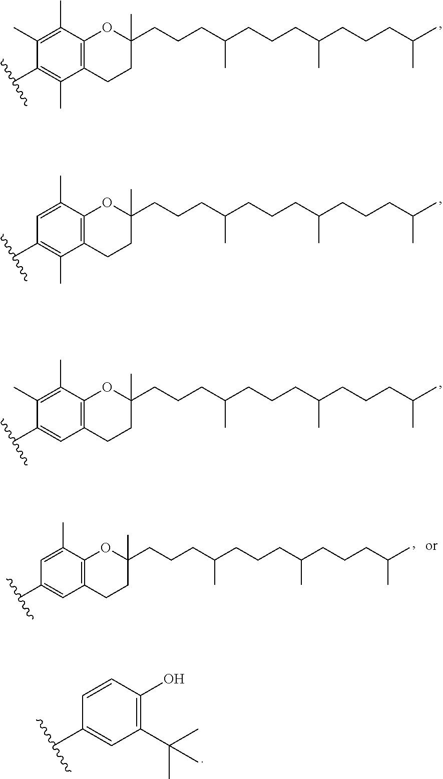 [SMILES: *c1c(C)c(C)c2c(c1C)CCC(C)(CCCC(C)CCCC(C)CCCC(C)C)O2 ; *c1cc(C)c2c(c1C)CCC(C)(CCCC(C)CCCC(C)CCCC(C)C)O2 ; *c1cc2c(c(C)c1C)OC(C)(CCCC(C)CCCC(C)CCCC(C)C)CC2 ; *c1cc(C)c2c(c1)CCC(C)(CCCC(C)CCCC(C)CCCC(C)C)O2 ; *c1ccc(O)c(C(C)(C)C)c1]

In certain embodiments, X is of the formula:

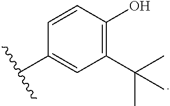 [SMILES: *c1ccc(O)c(C(C)(C)C)c1]

In certain embodiments, is:

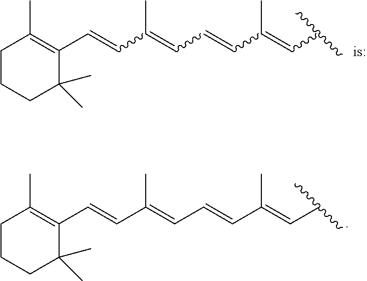

In certain embodiments, is:

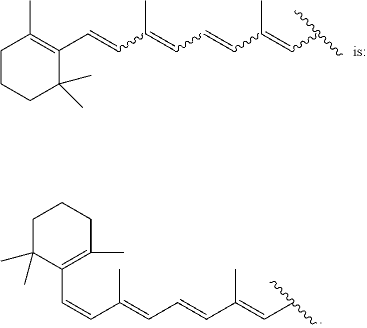

In certain embodiments, is:

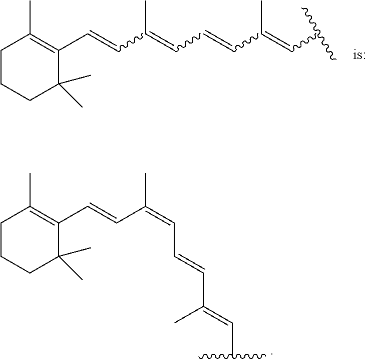

In certain embodiments, is:

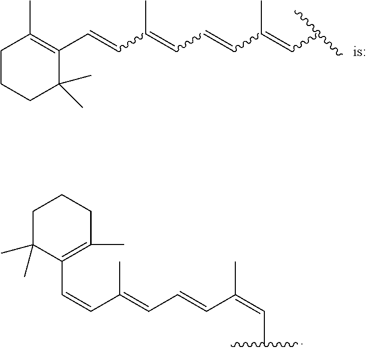

In certain embodiments, is:

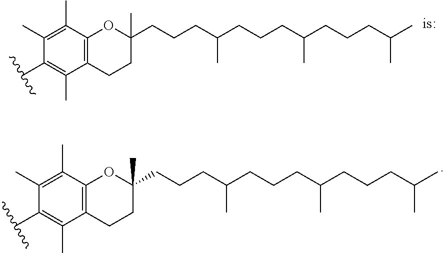

In certain embodiments, is:

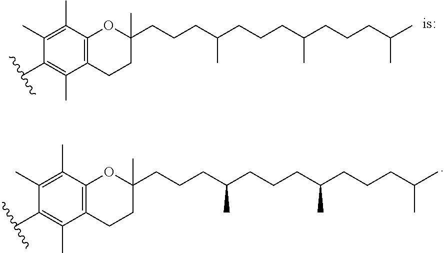

In certain embodiments, is:

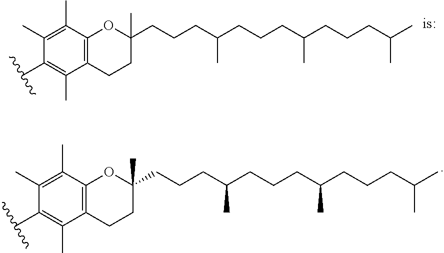

In certain embodiments, is:

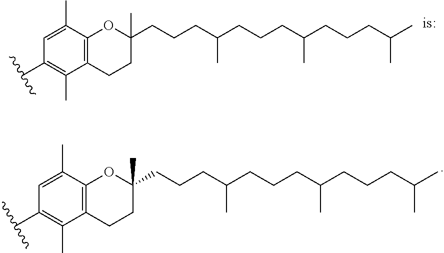 [SMILES: *c1cc(C)c2c(c1C)CCC(C)(CCCC(C)CCCC(C)CCCC(C)C)O2 ; *c1cc(C)c2c(c1C)CC[C@@](C)(CCCC(C)CCCC(C)CCCC(C)C)O2]

In certain embodiments, is:

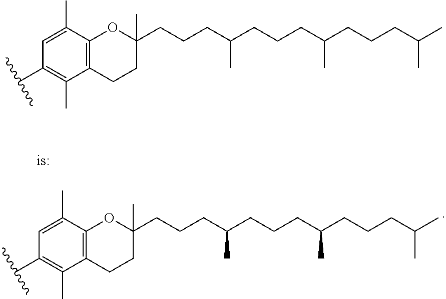 [SMILES: *c1cc(C)c2c(c1C)CCC(C)(CCCC(C)CCCC(C)CCCC(C)C)O2 ; *c1cc(C)c2c(c1C)CCC(C)(CCC[C@H](C)CCC[C@H](C)CCCC(C)C)O2]

In certain embodiments, is:

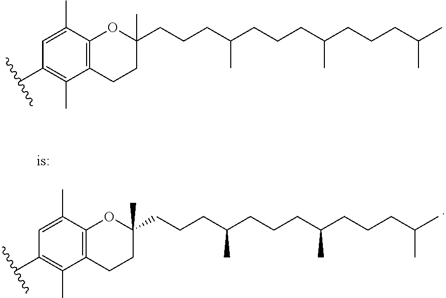 [SMILES: *c1cc(C)c2c(c1C)CCC(C)(CCCC(C)CCCC(C)CCCC(C)C)O2 ; *c1cc(C)c2c(c1C)CC[C@@](C)(CCC[C@H](C)CCC[C@H](C)CCCC(C)C)O2]

In certain embodiments, is:

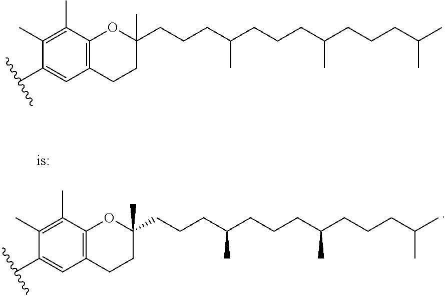 [SMILES: *c1cc2c(c(C)c1C)OC(C)(CCCC(C)CCCC(C)CCCC(C)C)CC2 ; *c1cc2c(c(C)c1C)O[C@](C)(CCC[C@H](C)CCC[C@H](C)CCCC(C)C)CC2]

In certain embodiments, is:

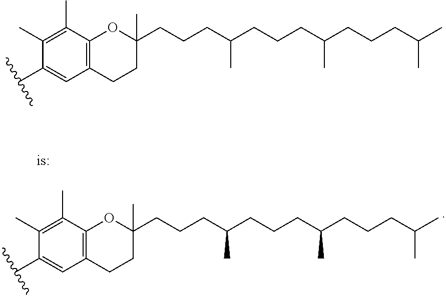 [SMILES: *c1cc2c(c(C)c1C)OC(C)(CCCC(C)CCCC(C)CCCC(C)C)CC2 ; *c1cc2c(c(C)c1C)OC(C)(CCC[C@H](C)CCC[C@H](C)CCCC(C)C)CC2]

In certain embodiments, is:

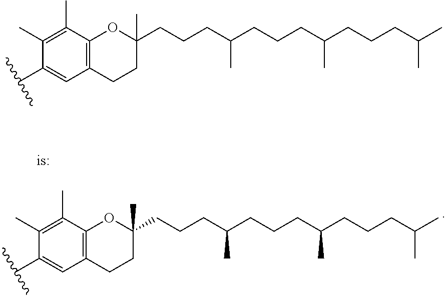 [SMILES: *c1cc2c(c(C)c1C)OC(C)(CCCC(C)CCCC(C)CCCC(C)C)CC2 ; *c1cc2c(c(C)c1C)O[C@](C)(CCC[C@H](C)CCC[C@H](C)CCCC(C)C)CC2]

In certain embodiments, is:

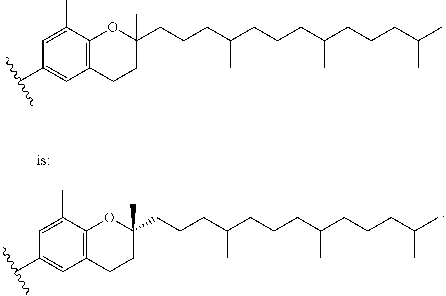 [SMILES: *c1cc(C)c2c(c1)CCC(C)(CCCC(C)CCCC(C)CCCC(C)C)O2 ; *c1cc(C)c2c(c1)CC[C@@](C)(CCCC(C)CCCC(C)CCCC(C)C)O2]

In certain embodiments, is:

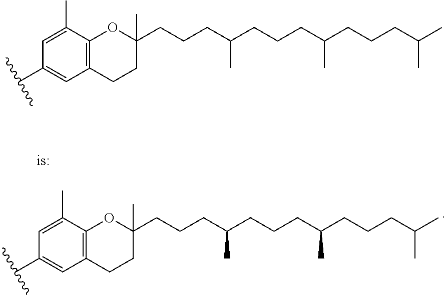 [SMILES: *c1cc(C)c2c(c1)CCC(C)(CCCC(C)CCCC(C)CCCC(C)C)O2 ; *c1cc(C)c2c(c1)CCC(C)(CCC[C@H](C)CCC[C@H](C)CCCC(C)C)O2]

In certain embodiments, is:

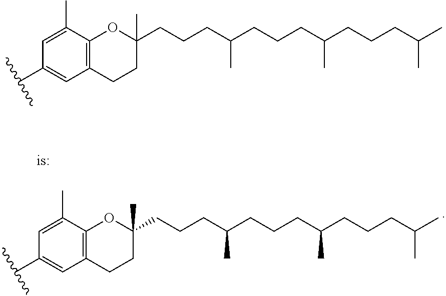 [SMILES: *c1cc(C)c2c(c1)CCC(C)(CCCC(C)CCCC(C)CCCC(C)C)O2 ; *c1cc(C)c2c(c1)CC[C@@](C)(CCC[C@H](C)CCC[C@H](C)CCCC(C)C)O2]

In certain embodiments, the ester is of Formula (1):

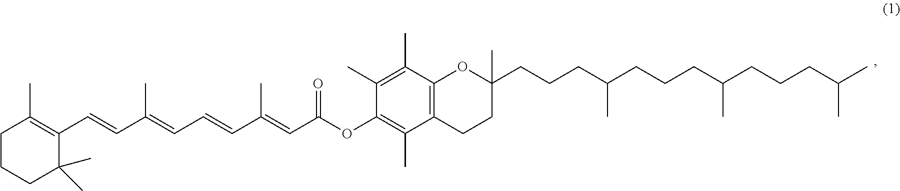 [SMILES: CC1=C(/C=C/C(C)=C/C=C/C(C)=C/C(=O)Oc2c(C)c(C)c3c(c2C)CCC(C)(CCCC(C)CCCC(C)CCCC(C)C)O3)C(C)(C)CCC1]

or a tautomer, isotopically labeled compound, solvate, polymorph, or co-crystal thereof. In certain embodiments, the ester is of Formula (1), or a tautomer or isotopically labeled compound thereof. In certain embodiments, the ester is of Formula (1), or a tautomer thereof. In certain embodiments, Formula (1) is:

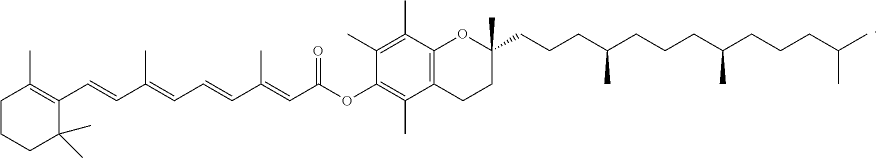 [SMILES: CC1=C(/C=C/C(C)=C/C=C/C(C)=C/C(=O)Oc2c(C)c(C)c3c(c2C)CC[C@@](C)(CCC[C@H](C)CCC[C@H](C)CCCC(C)C)O3)C(C)(C)CCC1]

In certain embodiments, the ester is of Formula (2):

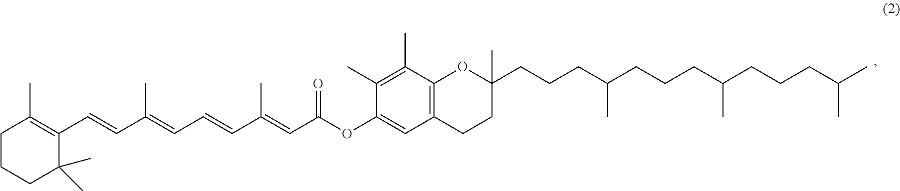 [SMILES: CC1=C(/C=C/C(C)=C/C=C/C(C)=C/C(=O)Oc2cc3c(c(C)c2C)OC(C)(CCCC(C)CCCC(C)CCCC(C)C)CC3)C(C)(C)CCC1]

or a tautomer, isotopically labeled compound, solvate, polymorph, or co-crystal thereof. In certain embodiments, the ester is of Formula (2), or a tautomer or isotopically labeled compound thereof. In certain embodiments, the ester is of Formula (2), or a tautomer thereof. In certain embodiments, Formula (2) is:

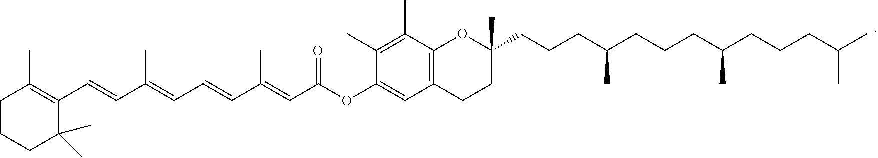 [SMILES: CC1=C(/C=C/C(C)=C/C=C/C(C)=C/C(=O)Oc2cc3c(c(C)c2C)O[C@](C)(CCC[C@H](C)CCC[C@H](C)CCCC(C)C)CC3)C(C)(C)CCC1]

In certain embodiments, the ester is of Formula (3):

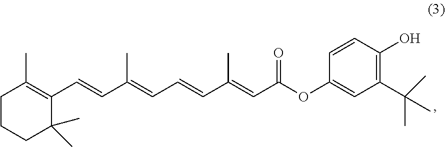 [SMILES: CC1=C(/C=C/C(C)=C/C=C/C(C)=C/C(=O)Oc2ccc(O)c(C(C)(C)C)c2)C(C)(C)CCC1]

or a tautomer, isotopically labeled compound, solvate, polymorph, or co-crystal thereof. In certain embodiments, the ester is of Formula (3), or a tautomer or isotopically labeled compound thereof. In certain embodiments, the ester is of Formula (3), or a tautomer thereof.

In certain embodiments, the ester is of Formula (4):

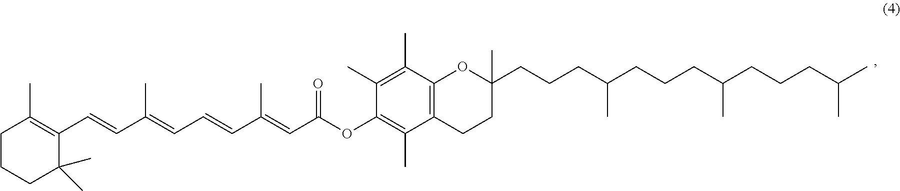 [SMILES: CC1=C(/C=C/C(C)=C/C=C/C(C)=C/C(=O)Oc2c(C)c(C)c3c(c2C)CCC(C)(CCCC(C)CCCC(C)CCCC(C)C)O3)C(C)(C)CCC1]

or a tautomer, isotopically labeled compound, solvate, polymorph, or co-crystal thereof. In certain embodiments, the ester is of Formula (4), or a tautomer or isotopically labeled compound thereof. In certain embodiments, the ester is of Formula (4), or a tautomer thereof. In certain embodiments, Formula (4) is:

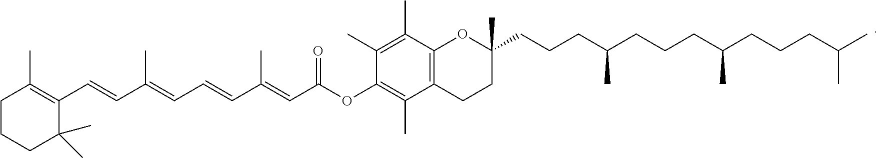 [SMILES: CC1=C(/C=C/C(C)=C/C=C/C(C)=C/C(=O)Oc2c(C)c(C)c3c(c2C)CC[C@@](C)(CCC[C@H](C)CCC[C@H](C)CCCC(C)C)O3)C(C)(C)CCC1]

In certain embodiments, the ester is of Formula (5):

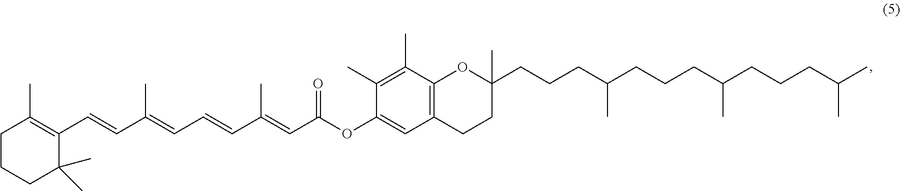 [SMILES: CC1=C(/C=C/C(C)=C/C=C/C(C)=C/C(=O)Oc2cc3c(c(C)c2C)OC(C)(CCCC(C)CCCC(C)CCCC(C)C)CC3)C(C)(C)CCC1]

or a tautomer, isotopically labeled compound, solvate, polymorph, or co-crystal thereof. In certain embodiments, the ester is of Formula (5), or a tautomer or isotopically labeled compound thereof. In certain embodiments, the ester is of Formula (5), or a tautomer thereof. In certain embodiments, Formula (5) is:

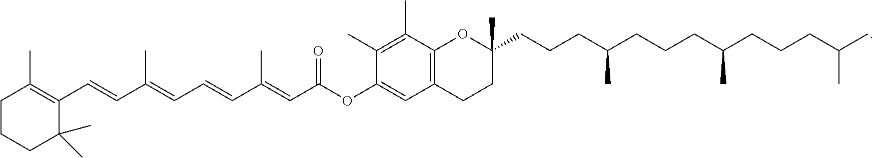 [SMILES: CC1=C(/C=C/C(C)=C/C=C/C(C)=C/C(=O)Oc2cc3c(c(C)c2C)O[C@](C)(CCC[C@H](C)CCC[C@H](C)CCCC(C)C)CC3)C(C)(C)CCC1]

In certain embodiments, the ester, or a tautomer, isotopically labeled compound, solvate, polymorph, or co-crystal thereof, is a substantially racemic mixture of isomers (e.g., E/Z and/or stereoisomers). In certain embodiments, the molar concentration of most prevalent isomer in the mixture of isomers is between 40% and 50%, between 50% and 60%, between 60% and 70%, between 70% and 80%, between 80% and 90%, between 90% and 99%, or between 99% and 99.9%, inclusive.

In another aspect, the present disclosure provides a method of producing the ester, or a tautomer, isotopically labeled compound, solvate, polymorph, or co-crystal thereof, the method comprising incubating a second reaction mixture for a second time duration sufficient to produce the ester, tautomer, isotopically labeled compound, solvate, polymorph, or co-crystal, wherein the second reaction mixture comprises:

(a) an anhydride of Formula (B):

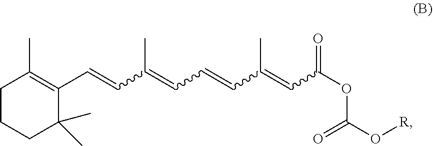

or a tautomer, isotopically labeled compound, solvate, polymorph, or co-crystal thereof, wherein R is substituted or unsubstituted alkyl;

(b) an alcohol of Formula (C):

HO—X (C),

or a tautomer, isotopically labeled compound, solvate, polymorph, or co-crystal thereof;

(c) a second solvent;

(d) optionally a second base; and

(e) optionally an esterification catalyst.

In certain embodiments, Formula (B) is of the formula:

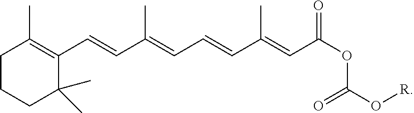

In certain embodiments, Formula (C) is Formula (C1):

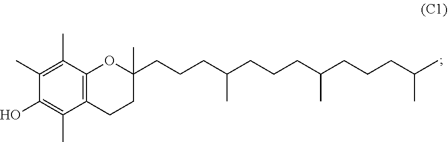 [SMILES: Cc1c(C)c2c(c(C)c1O)CCC(C)(CCCC(C)CCCC(C)CCCC(C)C)O2]

and

Formula (I) is Formula (I1):

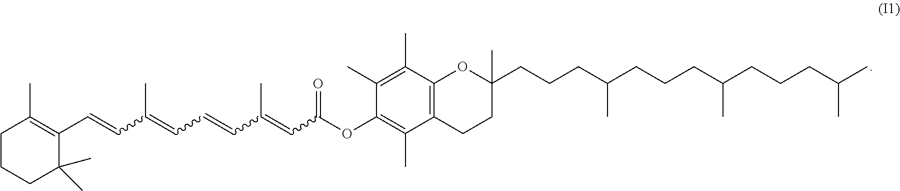 [SMILES: CC(C=CC1=C(C)CCCC1(C)C)=CC=CC(C)=CC(=O)Oc1c(C)c(C)c2c(c1C)CCC(C)(CCCC(C)CCCC(C)CCCC(C)C)O2]

In certain embodiments, Formula (C) or (C1) is of the formula:

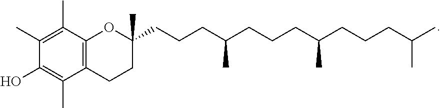 [SMILES: Cc1c(C)c2c(c(C)c1O)CC[C@@](C)(CCC[C@H](C)CCC[C@H](C)CCCC(C)C)O2]

In certain embodiments, Formula (C) is Formula (C2):

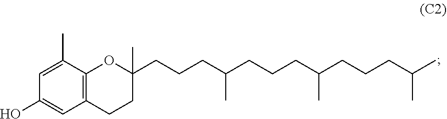 [SMILES: Cc1cc(O)cc2c1OC(C)(CCCC(C)CCCC(C)CCCC(C)C)CC2]

and

Formula (I) is Formula (I2):

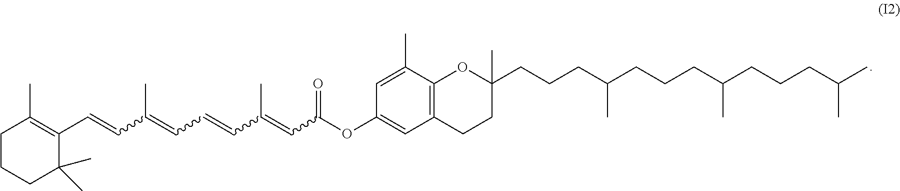 [SMILES: CC(C=CC1=C(C)CCCC1(C)C)=CC=CC(C)=CC(=O)Oc1cc(C)c2c(c1)CCC(C)(CCCC(C)CCCC(C)CCCC(C)C)O2]

In certain embodiments, Formula (C) or (C2) is of the formula:

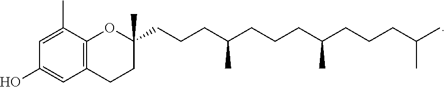 [SMILES: Cc1cc(O)cc2c1O[C@](C)(CCC[C@H](C)CCC[C@H](C)CCCC(C)C)CC2]

In certain embodiments, Formula (C) is Formula (C3):

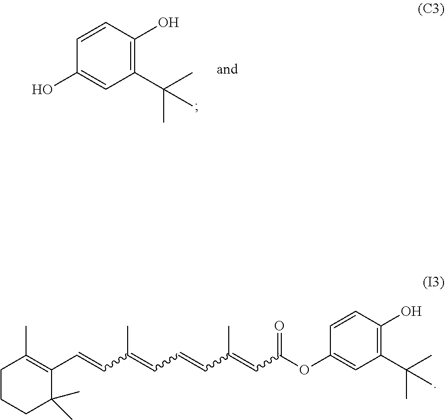 [SMILES: CC(C)(C)c1cc(O)ccc1O ; CC(C=CC1=C(C)CCCC1(C)C)=CC=CC(C)=CC(=O)Oc1ccc(O)c(C(C)(C)C)c1]

Formula (I) is Formula (I3):

In certain embodiments, Formula (C) is Formula (C4):

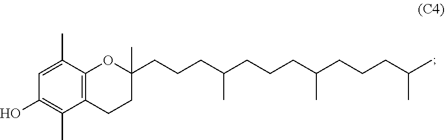 [SMILES: Cc1cc(O)c(C)c2c1OC(C)(CCCC(C)CCCC(C)CCCC(C)C)CC2]

and

Formula (I) is Formula (I4):

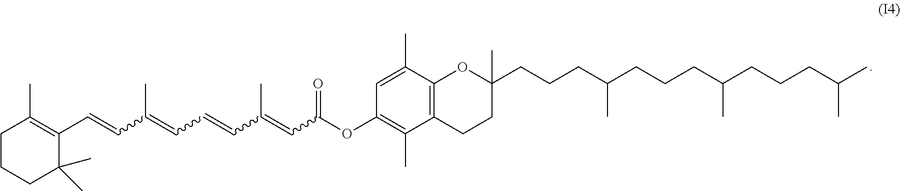 [SMILES: CC(C=CC1=C(C)CCCC1(C)C)=CC=CC(C)=CC(=O)Oc1cc(C)c2c(c1C)CCC(C)(CCCC(C)CCCC(C)CCCC(C)C)O2]

In certain embodiments, Formula (C) or (C4) is of the formula:

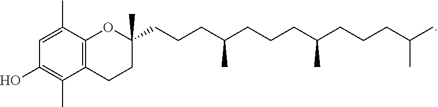 [SMILES: Cc1cc(O)c(C)c2c1O[C@](C)(CCC[C@H](C)CCC[C@H](C)CCCC(C)C)CC2]

In certain embodiments, Formula (C) is Formula (C5):

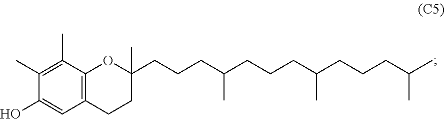 [SMILES: Cc1c(O)cc2c(c1C)OC(C)(CCCC(C)CCCC(C)CCCC(C)C)CC2]

and

Formula (I) is Formula (15):

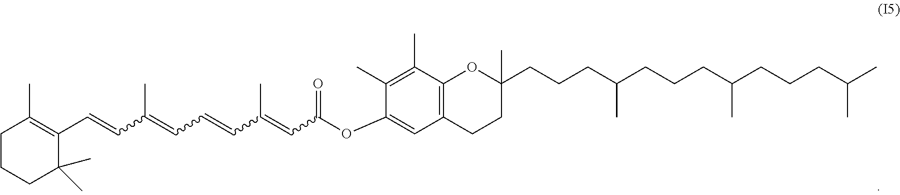 [SMILES: CC(C=CC1=C(C)CCCC1(C)C)=CC=CC(C)=CC(=O)Oc1cc2c(c(C)c1C)OC(C)(CCCC(C)CCCC(C)CCCC(C)C)CC2]

In certain embodiments, Formula (C) or (C5) is of the formula:

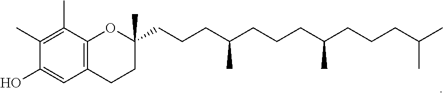 [SMILES: Cc1c(O)cc2c(c1C)O[C@](C)(CCC[C@H](C)CCC[C@H](C)CCCC(C)C)CC2]

In certain embodiments, the anhydride of Formula (B), or a tautomer, isotopically labeled compound, solvate, polymorph, or co-crystal thereof, is substantially a single isomer (e.g., E/Z and stereoisomer). In certain embodiments, the anhydride of Formula (B), or a tautomer, isotopically labeled compound, solvate, polymorph, or co-crystal thereof, is a mixture of isomers (e.g., E/Z isomers and/or stereoisomers), and the molar concentration of the most prevalent isomer (e.g., the isomer with fully retained isomerism with respect to the acid of Formula (A1), or a tautomer, isotopically labeled compound, salt, solvate, polymorph, or co-crystal thereof) in the mixture of isomers is between 40% and 50%, between 50% and 60%, between 60% and 70%, between 70% and 80%, between 80% and 90%, between 90% and 99%, or between 99% and 99.9%, inclusive.

In certain embodiments, the molar ratio of the amount of (a) of the second reaction mixture to the amount of (b) of the second reaction mixture is between 1:0.1 and 1:0.3, between 1:0.3 and 1:0.75, between 1:0.75 and 1:1, between 1:1 and 1:1.25, between 1:1.25 and 1:3, or between 1:3 and 1:10, inclusive. In certain embodiments, the molar ratio of the amount of (a) of the second reaction mixture to the amount of (b) of the second reaction mixture is between 1:0.3 to 1:3, inclusive. In certain embodiments, the molar ratio of the amount of (a) of the second reaction mixture to the amount of (b) of the second reaction mixture is between 1:0.75 to 1:2, inclusive. In certain embodiments, the molar ratio of the amount of (a) of the second reaction mixture to the amount of (b) of the second reaction mixture is between 1:0.75 to 1:1.25, inclusive.

In certain embodiments, the second solvent is substantially one single solvent. In certain embodiments, the second solvent is a mixture of two or more (e.g., three) solvents (e.g., solvents described in this paragraph). In certain embodiments, the second solvent is an organic solvent. In certain embodiments, the second solvent is an aprotic solvent. In certain embodiments, the second solvent is an ether solvent. In certain embodiments, the second solvent is a ketone solvent. In certain embodiments, the second solvent is an alkane solvent. In certain embodiments, the second solvent is an alcohol solvent. In certain embodiments, the second solvent is an aromatic organic solvent. In certain embodiments, the second solvent is benzene, toluene, o-xylene, m-xylene, or p-xylene, or a mixture thereof. In certain embodiments, the second solvent is a non-aromatic organic solvent. In certain embodiments, the second solvent is acetonitrile, dioxane, or tetrahydrofuran, or a mixture thereof. In certain embodiments, the second solvent is acetonitrile. In certain embodiments, the first solvent is acetone, chloroform, dichloromethane, diethyl ether, ethyl acetate, methyl tert-butyl ether, or 2-methyltetrahydrofuran, or a mixture thereof. In certain embodiments, the second solvent is an inorganic solvent. In certain embodiments, the boiling point of the second solvent at about 1 atm is between 30 and 50, between 50 and 70, between 70 and 100, between 100 and 130, between 130 and 160, or between 160 and 200° C., inclusive.

In certain embodiments, the second base, if present, is an organic base. In certain embodiments, the second base, if present, is a mono-, di-, or tri-(unsubstituted C₁₋₆ alkyl) amine. In certain embodiments, the second base, if present, is a tri-(unsubstituted C₁₋₆ alkyl) amine. In certain embodiments, the second base, if present, is trimethylamine, triethylamine, or N,N-diisopropylethylamine, or a mixture thereof. In certain embodiments, the second base, if present, is a cyclic non-aromatic amine. In certain embodiments, the second base, if present, is an aromatic amine (e.g., pyridine). In certain embodiments, the second base, if present, is an inorganic base. In certain embodiments, the second base, if present, is Li₂CO₃, Na₂CO₃, or K₂CO₃, or a mixture thereof. In certain embodiments, the second base, if present, is LiHCO₃, NaHCO₃, or KHCO₃, or a mixture thereof. In certain embodiments, the second base, if present, is ammonia, ammonium carbonate, or ammonium hydroxide.

In certain embodiments, the molar ratio of the amount of the second base, if present, to the amount of (b) of the second reaction mixture is between 1:1 and 2:1, between 2:1 and 3:1, between 3:1 and 5:1, or between 5:1 and 10:1, inclusive. In certain embodiments, the molar ratio of the amount of the second base, if present, to the amount of (b) of the second reaction mixture is between 1:1 and 5:1, inclusive.

In certain embodiments, the esterification catalyst, if present, is a Yamaguchi esterification catalyst. In certain embodiments, the esterification catalyst, if present, is 4-dimethylaminopyridine, or a salt or solvate thereof. In certain embodiments, the esterification catalyst, if present, is 4-(pyrrolidin-1-yl)pyridine, or a salt or solvate thereof. In certain embodiments, the esterification catalyst, if present, is pyridine, or a salt thereof.

In certain embodiments, the molar ratio of the amount of the esterification catalyst, if present, to the amount of (b) of the second reaction mixture is between 0.01:1 and 0.03:1, between 0.03:1 and 0.1:1, between 0.1:1 and 0.3:1, between 0.3:1 and 1:1, between 1:1 and 2:1, or between 2:1 and 5:1, inclusive. In certain embodiments, the molar ratio of the amount of the esterification catalyst, if present, to the amount of (b) of the second reaction mixture is between 0.1:1 to 2:1, inclusive. In certain embodiments, the molar ratio of the amount of the esterification catalyst, if present, to the amount of (b) of the second reaction mixture is between 0.03:1 to 1:1, inclusive.

In certain embodiments, the second reaction mixture is substantially free of water (e.g., H₂O, HDO, or D₂O, or a mixture thereof). In certain embodiments, the second reaction mixture is between 95% and 97%, between 97% and 99%, between 99% and 99.9%, or between 99.9% and 99.99%, by weight, free of water.

In certain embodiments, the temperature of the second reaction mixture is between −20 and 0, between 0 and 20, between 20 and 40, between 40 and 60, between 60 and 80, or between 80 and 100° C., inclusive. In certain embodiments, the temperature of the second reaction mixture is between 0 and 60° C., inclusive. In certain embodiments, the temperature of the second reaction mixture is between 20 and 60° C., inclusive. In certain embodiments, the temperature of the second reaction mixture is between 30 and 60° C., inclusive. In certain embodiments, the temperature of the second reaction mixture is substantially constant over the second time duration.

In certain embodiments, the second time duration is between 1 and 10 minutes, between 10 and 60 minutes, between 1 and 3 hours, between 3 and 6 hours, between 6 and 12 hours, between 12 and 24 hours, or between 1 and 3 days, inclusive. In certain embodiments, the second time duration is between 10 minutes and 1 day, inclusive. In certain embodiments, the second time duration is between 20 minutes and 3 hours, inclusive.

In certain embodiments, the rate of conversion of (b) of the second reaction mixture to the ester, or a tautomer, isotopically labeled compound, solvate, polymorph, or co-crystal thereof, is between 10% and 20%, between 20% and 40%, between 40% and 60%, between 60% and 80%, or between 80% and 99%, inclusive. In certain embodiments, the rate of conversion of (b) of the second reaction mixture to the ester, or a tautomer, isotopically labeled compound, solvate, polymorph, or co-crystal thereof, is between 10% and 99%, inclusive. In certain embodiments, the rate of conversion of (b) of the second reaction mixture to the ester, or a tautomer, isotopically labeled compound, solvate, polymorph, or co-crystal thereof, is between 40% and 70%, inclusive.

In certain embodiments, the method further comprises incubating a third reaction mixture comprising the second reaction mixture and ethanolamine, or a salt thereof, for a third time duration.

In certain embodiments, the molar ratio of the amount of ethanolamine, or a salt thereof, to the amount of (b) of the second reaction mixture is between 0.1:1 and 1:1, between 1:1 and 10:1, between 10:1 and 100:1, or between 100:1 and 1,000:1, inclusive. In certain embodiments, the molar ratio of the amount of ethanolamine, or a salt thereof, to the amount of (b) of the second reaction mixture is between 1:1 and 100:1, inclusive.

In certain embodiments, the temperature of the third reaction mixture is between −20 and 0, between 0 and 20, between 20 and 40, between 40 and 60, between 60 and 80, or between 80 and 100° C., inclusive. In certain embodiments, the temperature of the third reaction mixture is between 0 and 60° C., inclusive. In certain embodiments, the temperature of the third reaction mixture is between 20 and 60° C., inclusive. In certain embodiments, the temperature of the third reaction mixture is between 30 and 60° C., inclusive. In certain embodiments, the temperature of the third reaction mixture is substantially constant over the third time duration.

In certain embodiments, the third time duration is between 1 and 10 minutes, between 10 and 60 minutes, between 1 and 3 hours, between 3 and 6 hours, between 6 and 12 hours, between 12 and hours, or between 1 and 3 days, inclusive. In certain embodiments, the third time duration is between 10 minutes and 1 day, inclusive. In certain embodiments, the third time duration is between 10 minutes and 1.5 hours, inclusive.

In certain embodiments, the third reaction mixture is substantially free of water (e.g., H₂O, HDO, or D₂O, or a mixture thereof). In certain embodiments, the third reaction mixture is between 95% and 97%, between 97% and 99%, between 99% and 99.9%, or between 99.9% and 99.99%, by weight, free of water.

In certain embodiments, the step of incubating the third reaction mixture is immediately after the step of incubating the second reaction mixture.

In certain embodiments, the method further comprises purifying the ester, or a tautomer, isotopically labeled compound, solvate, polymorph, or co-crystal thereof. In certain embodiments, the step of purifying the ester, or a tautomer, isotopically labeled compound, solvate, polymorph, or co-crystal thereof, comprises liquid-liquid phase separation, drying, filtration, concentration, chromatography, decolorization, or recrystallization, or a combination thereof. In certain embodiments, the liquid-liquid phase separation is a separation of an organic liquid phase and an aqueous phase. In certain embodiments, the drying is drying an organic liquid phase over a solid drying agent (e.g., anhydrous Na₂SO₄, anhydrous MgSO₄, anhydrous CaSO₄, anhydrous CaCl₂), or activated molecular sieves). In certain embodiments, the filtration is a filtration of a mixture of a liquid phase (e.g., organic liquid phase) and a solid drying agent, or hydrates thereof, to remove the solid drying agent, or the hydrates thereof. In certain embodiments, the concentration is concentration of a liquid phase (e.g., organic liquid phase) to remove part or substantially all of the volatiles (e.g., organic solvents). In certain embodiments, the concentration is performed under a pressure lower than 1 atm (e.g., between 0.001 and 0.01, between 0.01 and 0.1, or between 0.1 and 1 atm, inclusive). In certain embodiments, the concentration is performed under a temperature of between 0 and 10, between 10 and 20, between 20 and 25, between 25 and 35, between 35 and 50, or between 50 and 80° C., inclusive. In certain embodiments, the chromatography is flash chromatography (e.g., normal-phase flash chromatography (e.g., over silica gel)). In certain embodiments, the chromatography is high-performance liquid chromatography (HPLC) (e.g., reverse-phase HPLC or normal-phase HPLC). In certain embodiments, the decolorization comprises redissolving in an organic solvent, decolorization, and concentration. In certain embodiments, the decolorization comprises contacting with a solid decolorization agent (e.g., activated charcoal). In certain embodiments, the recrystallization is a single-solvent recrystallization. In certain embodiments, the recrystallization is a multi-solvent (e.g., bi-solvent or tri-solvent) recrystallization.

In certain embodiments, the recrystallization is a hot filtration-recrystallization. In certain embodiments, the step of purifying the ester, or a tautomer, isotopically labeled compound, solvate, polymorph, or co-crystal thereof, further comprises removing part or substantially all of the volatiles (e.g., organic solvents) by decreasing the pressure (e.g., to a pressure of lower than 1 atm (e.g., between 0.001 and 0.01, between 0.01 and 0.1, or between 0.1 and 1 atm, inclusive) and/or increasing the temperature (e.g., to a temperature between 25 and 35, between 35 and 50, or between and 80° C., inclusive).

In certain embodiments, in the reaction mixture (e.g., the second or third rection mixture) immediately before the step of purifying the ester, or a tautomer, isotopically labeled compound, solvate, polymorph, or co-crystal thereof, the molar concentration of the ester, or a tautomer, isotopically labeled compound, solvate, polymorph, or co-crystal thereof, in all compounds that are present in the reaction mixture and comprise a moiety of the formula:

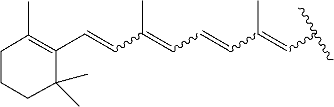

is between 40% and 50%, between 50% and 60%, between 60% and 70%, between 70% and 80%, between 80% and 90%, between 90% and 99%, or between 99% and 99.9%, inclusive.

In certain embodiments, in the reaction mixture (e.g., the second or third reaction mixture) immediately before the step of purifying the ester, or a tautomer, isotopically labeled compound, solvate, polymorph, or co-crystal thereof, the ester, or a tautomer, isotopically labeled compound, solvate, polymorph, or co-crystal thereof, is a mixture of isomers (e.g., E/Z isomers and/or stereoisomers), and the molar concentration of the most prevalent isomer (e.g., the isomer with fully retained isomerism with respect to the anhydride of Formula (B), or a tautomer, isotopically labeled compound, solvate, polymorph, or co-crystal thereof, and the alcohol of Formula (C), or a tautomer, isotopically labeled compound, solvate, polymorph, or co-crystal thereof) in the mixture of isomers is between 40% and 50%, between 50% and 60%, between 60% and 70%, between 70% and 80%, between 80% and 90%, between 90% and 99%, or between 99% and 99.9%, inclusive.

In certain embodiments, the method further comprises incubating a first reaction mixture for a first time duration sufficient to produce (a) of the second reaction mixture, wherein:

the first reaction mixture comprises:
(a) an acid of Formula (A1):

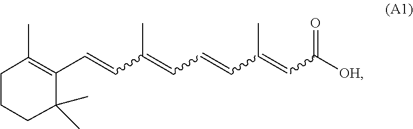 [SMILES: CC(C=CC1=C(C)CCCC1(C)C)=CC=CC(C)=CC(=O)O]

or a tautomer, isotopically labeled compound, salt, solvate, polymorph, or co-crystal thereof; (b) a chloroformate of Formula (A2):

Cl—C(═O)—O—R (A2),

or an isotopically labeled compound, solvate, polymorph, or co-crystal thereof, wherein R is substituted or unsubstituted alkyl;
(c) a first solvent; and
(d) optionally a first base; and

the step of incubating a first reaction mixture is prior to the step of incubating the second reaction mixture.

In certain embodiments, (a) of the first reaction mixture is of the formula:

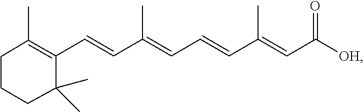 [SMILES: CC1=C(/C=C/C(C)=C/C=C/C(C)=C/C(=O)O)C(C)(C)CCC1]

or a tautomer, isotopically labeled compound, salt, solvate, polymorph, or co-crystal thereof (e.g., a tautomer, isotopically labeled compound, or salt thereof).

In certain embodiments, (a) of the first reaction mixture is of the formula:

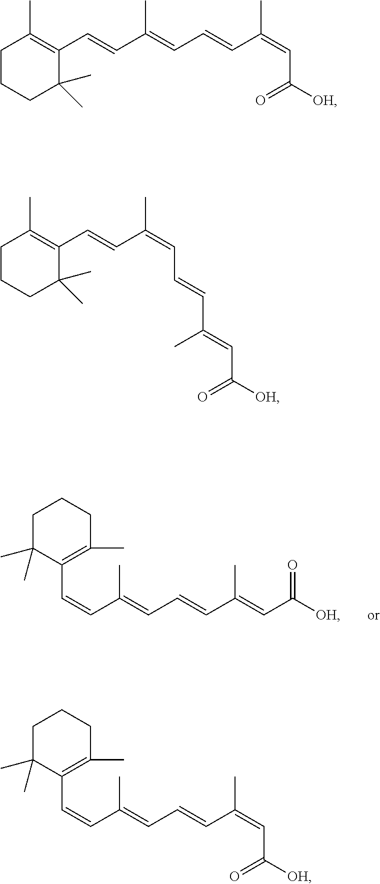 [SMILES: CC1=C(/C=C/C(C)=C/C=C/C(C)=C\C(=O)O)C(C)(C)CCC1 ; CC1=C(/C=C/C(C)=C\C=C\C(C)=C\C(=O)O)C(C)(C)CCC1 ; CC1=C(\C=C/C(C)=C/C=C/C(C)=C/C(=O)O)C(C)(C)CCC1 ; CC1=C(\C=C/C(C)=C/C=C/C(C)=C\C(=O)O)C(C)(C)CCC1]

or a tautomer, isotopically labeled compound, salt, solvate, polymorph, or co-crystal thereof (e.g., a tautomer, isotopically labeled compound, or salt thereof).

In certain embodiments, R is unsubstituted alkyl. In certain embodiments, R is unsubstituted C₁₋₆ alkyl. In certain embodiments, R is unsubstituted methyl. In certain embodiments, R is unsubstituted ethyl. In certain embodiments, R is unsubstituted propyl (e.g., n-Pr or i-Pr). In certain embodiments, R is unsubstituted butyl (e.g., n-Bu, sec-Bu, i-Bu, or t-Bu). In certain embodiments, R is unsubstituted ethyl. In certain embodiments, R is unsubstituted methyl, unsubstituted n-propyl, unsubstituted n-butyl, or unsubstituted isobutyl. In certain embodiments, R is substituted alkyl (e.g., alkyl substituted with one or more instances of halogen (e.g., F)). In certain embodiments, R is substituted C₁₋₆ alkyl. In certain embodiments, R is substituted methyl (e.g., fluorinated methyl or Bn). In certain embodiments, R is —CF₃. In certain embodiments, R is substituted ethyl, substituted propyl, or substituted butyl.

In certain embodiments, the molar ratio of the amount of (a) of the first reaction mixture to the amount of (b) of the first reaction mixture is between 1:0.3 and 1:0.5, between 1:0.5 and 1:0.67, between 1:0.67 and 1:1, between 1:1 and 1:1.2, between 1:1.2 and 1:2, between 1:2 and 1:3, inclusive. In certain embodiments, the molar ratio of the amount of (a) of the first reaction mixture to the amount of (b) of the first reaction mixture is between 1:1 to 1:1.2, inclusive. In certain embodiments, the molar ratio of the amount of (a) of the first reaction mixture to the amount of (b) of the first reaction mixture is between 1:0.67 to 1:1, inclusive.

In certain embodiments, the first solvent is substantially one single solvent. In certain embodiments, the first solvent is a mixture of two or more (e.g., three) solvents (e.g., solvents described in this paragraph). In certain embodiments, the first solvent is an organic solvent. In certain embodiments, the first solvent is an aprotic solvent. In certain embodiments, the first solvent is an ether solvent. In certain embodiments, the first solvent is a ketone solvent. In certain embodiments, the first solvent is an alkane solvent. In certain embodiments, the first solvent is an alcohol solvent. In certain embodiments, the first solvent is an aromatic organic solvent. In certain embodiments, the first solvent is benzene, toluene, o-xylene, m-xylene, or p-xylene, or a mixture thereof. In certain embodiments, the first solvent is a non-aromatic organic solvent. In certain embodiments, the first solvent is tetrahydrofuran, dichloromethane, or dioxane, or a mixture thereof. In certain embodiments, the first solvent is tetrahydrofuran. In certain embodiments, the first solvent is methyl tert-butyl ether or 2-methyltetrahydrofuran, or a mixture thereof. In certain embodiments, the first solvent is acetone, acetonitrile, chloroform, diethyl ether, or ethyl acetate, or a mixture thereof. In certain embodiments, the first solvent is an inorganic solvent. In certain embodiments, the boiling point of the first solvent at about 1 atm is between 30 and 50, between 50 and 70, between 70 and 100, between 100 and 130, between 130 and 160, or between 160 and 200° C., inclusive.

In certain embodiments, the first base, if present, is an organic base. In certain embodiments, the first base, if present, is a mono-, di-, or tri-(unsubstituted C₁₋₆ alkyl) amine. In certain embodiments, the first base, if present, is a tri-(unsubstituted C₁₋₆ alkyl) amine. In certain embodiments, the first base, if present, is trimethylamine, triethylamine, or N,N-diisopropylethylamine, or a mixture thereof. In certain embodiments, the first base, if present, is a cyclic non-aromatic amine. In certain embodiments, the first base, if present, is an aromatic amine (e.g., pyridine). In certain embodiments, the first base, if present, is trimethylamine, triethylamine, N,N-diisopropylethylamine, or pyridine. In certain embodiments, the first base, if present, is an inorganic base. In certain embodiments, the first base, if present, is Li₂CO₃, Na₂CO₃, or K₂CO₃, or a mixture thereof. In certain embodiments, the first base, if present, is LiHCO₃, NaHCO₃, or KHCO₃, or a mixture thereof. In certain embodiments, the first base, if present, is ammonia, ammonium carbonate, or ammonium hydroxide.

In certain embodiments, the molar ratio of the amount of the first base, if present, to the amount of (a) of the first reaction mixture is between 1:1 and 2:1, between 2:1 and 3:1, between 3:1 and 5:1, or between 5:1 and 10:1, inclusive. In certain embodiments, the molar ratio of the amount of the first base, if present, to the amount of (a) of the first reaction mixture is between 1:1 and 5:1, inclusive.

In certain embodiments, the temperature of the first reaction mixture is between −40 and −20, between −20 and 0, between 0 and 20, between 20 and 40, or between 40 and 60, inclusive. In certain embodiments, the temperature of the first reaction mixture is between −20 and 40° C., inclusive. In certain embodiments, the temperature of the first reaction mixture is between −20 and 20° C., inclusive. In certain embodiments, the temperature of the first reaction mixture is between 0 and 5° C., inclusive. In certain embodiments, the temperature of the first reaction mixture is substantially constant over the first time duration.

In certain embodiments, the first time duration is between 1 and 10 minutes, between 10 and 60 minutes, between 1 and 3 hours, between 3 and 6 hours, between 6 and 12 hours, between 12 and hours, or between 1 and 3 days, inclusive. In certain embodiments, the first time duration is between 10 minutes and 1 day, inclusive. In certain embodiments, the first time duration is between 20 minutes and 3 hours, inclusive.

In certain embodiments, the first reaction mixture is substantially free of water (e.g., H₂O, HDO, or D₂O, or a mixture thereof). In certain embodiments, the first reaction mixture is between 95% and 97%, between 97% and 99%, between 99% and 99.9%, or between 99.9% and 99.99%, by weight, free of water.

In certain embodiments, the pressure of the first, second, and third reaction mixtures is about 1 atm.

In certain embodiments, the rate of conversion of (a) of the first reaction mixture to (a) of the second reaction mixture is between 10% and 20%, between 20% and 40%, between 40% and 60%, between 60% and 80%, or between 80% and 99.9%, inclusive. In certain embodiments, rate of conversion of (a) of the first reaction mixture to (a) of the second reaction mixture is between 10% and 99.9%, inclusive. In certain embodiments, rate of conversion of (a) of the first reaction mixture to (a) of the second reaction mixture is between 50% and 99.9%, inclusive. In certain embodiments, rate of conversion of (a) of the first reaction mixture to (a) of the second reaction mixture is between 90% and 99.9%, inclusive.

In certain embodiments, the method further comprises purifying (a) of the second reaction mixture, wherein the step of purifying (a) of the second reaction mixture is prior to the step of incubating the second reaction mixture. In certain embodiments, the step of purifying (a) of the second reaction mixture comprises:

mixing the first reaction mixture with a non-polar organic solvent;

subsequently, filtration; and

subsequently and optionally, concentration.

In certain embodiments, the non-polar organic solvent is substantially one single solvent. In certain embodiments, the non-polar organic solvent is a mixture of two or more (e.g., three) solvents (e.g., solvents described in this paragraph). In certain embodiments, the non-polar organic solvent is an alkane solvent. In certain embodiments, the non-polar organic solvent is a pentane, a hexane, a heptane, or a petroleum ether, or a combination thereof. In certain embodiments, the non-polar organic solvent is an aromatic non-polar organic solvent. In certain embodiments, the non-polar organic solvent is benzene, toluene, o-xylene, m-xylene, or p-xylene, or a mixture thereof. In certain embodiments, the boiling point of the non-polar organic solvent at about 1 atm is between 30 and 50, between 50 and 70, between 70 and 100, between 100 and 130, between 130 and 160, or between and 200° C., inclusive. In certain embodiments, the filtration is a filtration of a mixture of a liquid phase and a solid phase to remove the solid phase (e.g., a salt of the first base, e.g., a HCl salt of the first base). In certain embodiments, the concentration is concentration of a liquid phase (e.g., organic liquid phase) to remove part or substantially all of the volatiles (e.g., organic solvents). In certain embodiments, the concentration is performed under a pressure lower than 1 atm (e.g., between 0.001 and 0.01, between 0.01 and 0.1, or between 0.1 and 1 atm, inclusive). In certain embodiments, the concentration is performed under a temperature of between 0 and 10, between 10 and 20, between 20 and 25, between 25 and 35, between 35 and 50, or between 50 and 80° C., inclusive. In certain embodiments, the method does not further comprise purifying (a) of the second reaction mixture prior to the step of incubating the second reaction mixture.

### Compositions and Kits

In another aspect, the present disclosure provides a composition comprising:

the ester, or a tautomer, isotopically labeled compound, solvate, polymorph, or co-crystal thereof; and

optionally an excipient.

In certain embodiments, the excipient, if present, is a cosmetically acceptable excipient. In certain embodiments, the composition is a cosmetic composition. In certain embodiments, the excipient, if present, is a pharmaceutically acceptable excipient. In certain embodiments, the composition is a pharmaceutical composition.

In certain embodiments, the composition further comprises an additional agent (e.g., additional cosmetic agent or additional pharmaceutical agent, or a combination thereof). The additional agent is different from the ester, or a tautomer, isotopically labeled compound, solvate, polymorph, or co-crystal thereof.

In certain embodiments, the composition is suitable for topical (e.g., by powders, ointments, creams, and/or drops) administration to a subject. In certain embodiments, the composition is suitable for oral administration to a subject. In certain embodiments, the composition is suitable for: enteral (e.g., oral), parenteral, intravenous, intramuscular, intra-arterial, intramedullary, intrathecal, subcutaneous, intraventricular, transdermal, intradermal, rectal, intravaginal, intraperitoneal, mucosal, nasal, buccal, or sublingual administration to a subject; administration to a subject by intratracheal instillation, bronchial instillation, and/or inhalation; and/or administration to a subject as an oral spray, nasal spray, and/or aerosol.

In certain embodiments, the subject is a mammal. In certain embodiments, the subject is a human. In certain embodiments, the subject is a human two-years and older. In certain embodiments, the subject is a human eighteen-years and older.

In another aspect, the present disclosure provides a composition comprising the ester, or a tautomer, isotopically labeled compound, solvate, polymorph, or co-crystal thereof, produced by the method.

The compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

The compositions can be prepared, packaged, and/or sold in bulk, as a single unit dose, and/or as a plurality of single unit doses. A “unit dose” is a discrete amount of the composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the excipient, and/or any additional ingredients in a composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj© 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

The compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of compositions provided herein are principally directed to compositions which are suitable for administration to humans, it will be understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a composition described herein including a compound described herein and an additional pharmaceutical agent shows a synergistic effect that is absent in a composition including one of the compound and the additional pharmaceutical agent, but not both.

The compound or composition can be administered concurrently with, prior to, or subsequent to one or more additional pharmaceutical agents, which are different from the compound or composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including surgery, radiation therapy, transplantation (e.g., stem cell transplantation, bone marrow transplantation), immunotherapy, and chemotherapy.

In another aspect, the present disclosure provides a kit comprising:

the ester, or a tautomer, isotopically labeled compound, solvate, polymorph, or co-crystal thereof, or the composition; and

optionally instructions for using the ester, tautomer, isotopically labeled compound, solvate, polymorph, co-crystal, or composition.

In certain embodiments, the kit comprises a first container, wherein the first container comprises the ester, or a tautomer, isotopically labeled compound, solvate, polymorph, or co-crystal thereof, or the composition. In some embodiments, the kit further comprises a second container. In certain embodiments, the second container comprises the instructions. In certain embodiments, the instructions comprise information required by a regulatory agency, such as the U.S. Food and Drug Administration (FDA) or European Medicines Agency (EMA). In certain embodiments, the instructions comprise prescribing information. In certain embodiments, the second container comprises the first container. In some embodiments, the kit further comprises a third container. In certain embodiments, the third container comprises the excipient. In certain embodiments, the third container comprises the additional agent. In certain embodiments, the second container comprises the third container. In certain embodiments, each of the first, second, and third containers is independently a vial, ampule, bottle, syringe, dispenser package, tube, or box.

### Methods of Use

In another aspect, the present disclosure provides a method of treating a skin disease in a subject in need thereof comprising administering to the subject in need thereof an effective amount of the ester, or a tautomer, isotopically labeled compound, solvate, polymorph, or co-crystal thereof, or the composition. In certain embodiments, the effective amount is a therapeutically effective amount.

In another aspect, the present disclosure provides a method of preventing a skin disease in a subject in need thereof comprising administering to the subject in need thereof an effective amount of the ester, or a tautomer, isotopically labeled compound, solvate, polymorph, or co-crystal thereof, or the composition. In certain embodiments, the effective amount is a prophylactically effective amount.

In certain embodiments, the skin disease is acantholysis, acne, acute febrile neutrophilic dermatosis, alopecia, anhidrosis, atrophic skin disease, autoimmune skin disease, Beare-Stevenson cutis gyrata syndrome, Behcet syndrome, blister, body odor, CEDNIK syndrome, cafe au lait spot, chilblain, congenital sensory neuropathy with anhidrosis, cutaneous edema, cutaneous fibrosis, cutaneous fistula, cutaneous leishmaniasis, cutaneous mastocytosis, dandruff, decubitus ulcer, dermal elastosis, dermatitis, dermatomyositis, dry skin, ectodermal dysplasia, Ehlers-Danlos syndrome, erythema, fish lymphocystis disease, genodermatosis, Graves dermopathy, Harada syndrome, Hoof disease, hyperhidrosis, ichthyosis, incontinentia pigmenti, jaundice, keloid, keratosis, lentigo, lichenoid dermatosis, lupus erythematosus, lupus verrucosus, MEDNIK syndrome, mammary gland disease, microphthalmia with linear skin defects syndrome, miliaria, mucinosis, nail disease, necrobiosis lipoidica, nephrogenic systemic fibrosis, neurocutaneous syndrome, neurotic excoriation, neutrophilic dermatosis, nevus, occupational skin disease, PAPA syndrome, pachydermoperiostosis, panniculitis, parapsoriasis, passive cutaneous anaphylaxis, photosensitivity disorders, pityriasis, pruritus, psitticine beak and feather disease, pustular skin disease, rhagades, rosacea, scale drop disease, scalp disease, scar, scleredema adultorum, scleredema neonatorum, scleroderma, seborrhea, severe dermatitis, multiple allergies and metabolic wasting syndrome, skin aging, skin appendage disease, skin depigmentation, skin desquamation, skin hyperpigmentation, skin hyperplasia, skin hyperproliferative disorders, skin hypopigmentation, skin infection, skin injury, skin irritation, skin lesion, skin necrosis, skin neoplasm, skin rash, skin striae, skin ulcer, steroid-induced atrophy, sweat gland disease, toxic epidermal necrolysis, Uasin Gishu disease, ulcer, urticaria, xanthomatosis, or xeroderma pigmentosum. In certain embodiments, the skin disease is acne. In certain embodiments, the skin disease is an autoimmune skin disease. In certain embodiments, the autoimmune skin disease is psoriasis. In certain embodiments, the skin disease is keratosis. In certain embodiments, the keratosis is wart. In certain embodiments, the skin disease is dermal elastosis, dry skin, seborrhea, rosacea, or lentigo.

In another aspect, the present disclosure provides a method of slowing the ageing of the skin or improving the appearance of the skin of a subject comprising administering to the subject an effective amount of the ester, or a tautomer, isotopically labeled compound, solvate, polymorph, or co-crystal thereof, or the composition. In certain embodiments, the effective amount is a cosmetically effective amount. In certain embodiments, the effective amount is effective in reducing wrinkles, lentigo, or warts in the skin or skin tags.

In another aspect, the present disclosure provides a method of modulating a retinoid receptor in a subject, biological sample, tissue, or cell comprising administering to the subject or contacting the biological sample, tissue, or cell with an effective amount of the ester, or a tautomer, isotopically labeled compound, solvate, polymorph, or co-crystal thereof, or the composition. In certain embodiments, the retinoid receptor is a retinoic acid receptor (e.g., retinoic acid receptor alpha, retinoic acid receptor beta, retinoic acid receptor gamma). In certain embodiments, the retinoid receptor is a retinoic acid receptor-related orphan receptor. In certain embodiments, the retinoid receptor is a retinoid X receptor. In certain embodiments, the effective amount is effective in activating the retinoid receptor in the subject, biological sample, tissue, or cell. In certain embodiments, the effective amount is effective in inhibiting the retinoid receptor in the subject, biological sample, tissue, or cell.

In certain embodiments, the administration is topical administration. In certain embodiments, the administration is oral administration. In certain embodiments, the administration is enteral (e.g., oral), parenteral, intravenous, intramuscular, intra-arterial, intramedullary, intrathecal, subcutaneous, intraventricular, transdermal, intradermal, rectal, intravaginal, intraperitoneal, mucosal, nasal, buccal, or sublingual administration; administration by intratracheal instillation, bronchial instillation, and/or inhalation; and/or administration as an oral spray, nasal spray, and/or aerosol.

In certain embodiments, the biological sample, tissue, or cell is in vitro. In certain embodiments, the biological sample, tissue, or cell is in vivo. the biological sample, tissue, or cell is ex vivo.

### EXAMPLES

In order that the invention described herein may be more fully understood, the following examples are set forth. The examples are offered to illustrate the methods and uses described herein and are not to be construed in any way as limiting their scope.

### Example 1. Synthesis of (±)α-tocopheryl retinoate

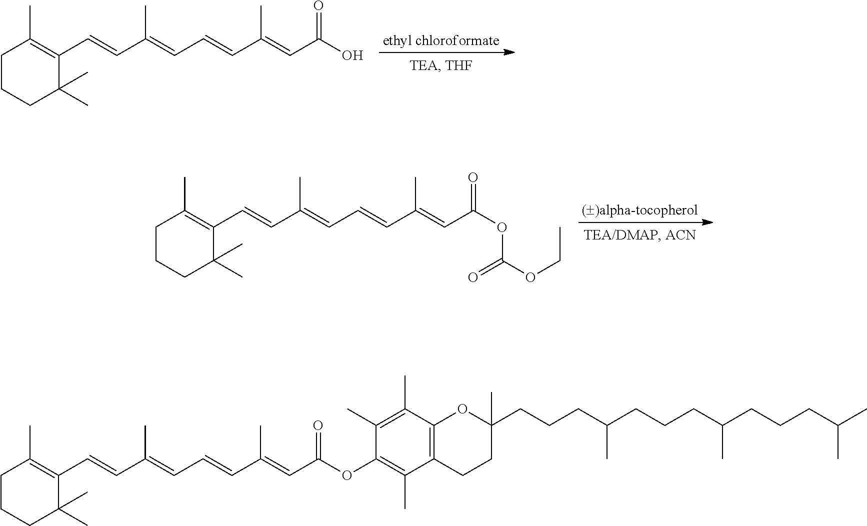

Retinoic acid (5.00 g, 16.64 mmol) was dissolved in anhydrous tetrahydrofuran (THF, 100 ml). Triethylamine (TEA, 5.0 ml) was added, and the mixture was stirred for five minutes. The solution of ethyl chloroformate (1.81 g, 16.64 mmol) in tetrahydrofuran (5 ml) was added dropwise at 0° C. The mixture was allowed to warm to room temperature and stirred for two hours.

Most of solvent was evaporated under reduced pressure. Hexane (50 ml) was added, and the mixture was stirred for five minutes. The triethylamine hydrochloride was collected by filtration. The filtrate was evaporated under reduced pressure.

The yellow residue was dissolved in anhydrous acetonitrile (ACN, 100 ml), and (±)α-tocopherol (7.16 g, 16.64 mmol) was added. After the mixture was stirred for five minutes, triethylamine (12.5 ml) and 4-dimethylaminopyridine (DMAP, 1.25 g) were added. The mixture was warmed to 50° C. and stirred for five hours.

Ethanolamine (5.0 ml) was added, and the mixture was stirred at 50° C. for 30 minutes.

Most of solvent was evaporated under reduced pressure, and the residue was mixed with ethyl acetate (200 ml). The ethyl acetate layer was washed with 1.0 N hydrochloride solution (200 ml), brine (200 ml), dried over magnesium sulfate, and evaporated under reduced pressure to give a crude product, which was purified by silica gel column with hexane/ethyl acetate as eluent to give 11.38 g of pale-yellow oil product. Molecular Formular: C₄₉H₇₆O₃. ¹H NMR: (400 MHz, DMSO-d₆): 0.75-0.87 (m, 12H), 0.93 (d, 6H), 0.97-1.05 (m, 2H), 1.01 (s, 3H), 1.06-1.13 (m, 2H), 1.15-1.30 (m, 12H), 1.48 (m, 3H), 1.57 (m, 2H), 1.69 (m, 2H), 1.93 (m, 3H), 1.97 (s, 3H), 1.99 (t, 3H), 2.00 (s, 3H), 2.01 (s, 6H), 2.02 (s, 3H), 2.33 (s, 3H), 2.56 (t, 2H), 6.21 (m, 2H), 6.29 (d, 1H), 6.53 (d, 1H), 7.15 (dd, 1H), 7.34 (s, 1H).

### Example 2. Synthesis of (+)δ-tocopheroyl retinoate

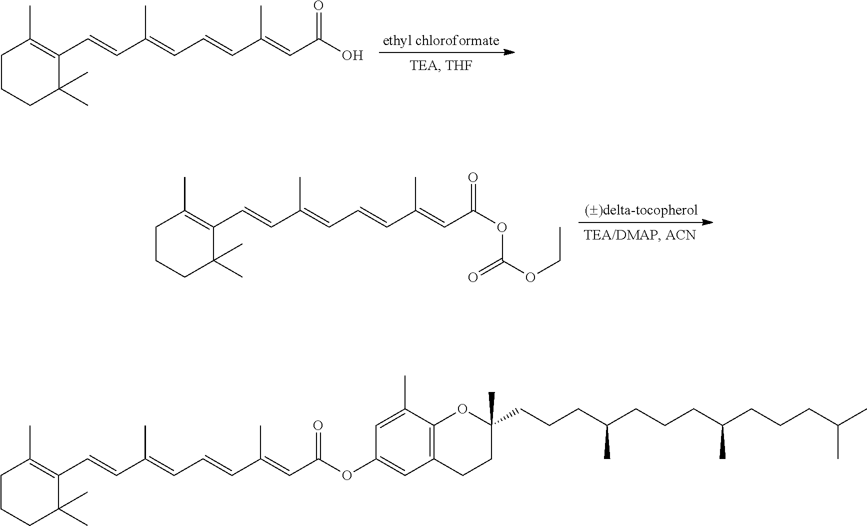

Retinoic acid (1.00 g, 3.33 mmol) was dissolved in anhydrous tetrahydrofuran (50 ml). Triethylamine (1.0 ml) was added, and the mixture was stirred for five minutes. The solution of ethyl chloroformate (0.36 g, 3.33 mmol) in tetrahydrofuran (1 ml) was added dropwise at 0° C. The mixture was allowed to warm to room temperature and stirred for two hours.

Hexane (50 ml) was added, and the triethylamine hydrochloride was collected by filtration. The filtrate was evaporated under reduced pressure.

The yellow residue was dissolved in anhydrous acetonitrile (50 ml), and (+)δ-tocopherol (1.49 g, 90%, 3.33 mmol) was added. After the mixture was stirred for five minutes, triethylamine (2.5 ml) and 4-dimethylaminopyridine (0.25 g) were added. The mixture was warmed to 50° C. and stirred for three hours.

Ethanolamine (1.0 ml) was added, and the mixture was stirred at 50° C. for 30 minutes. Most of solvent was evaporated under reduced pressure, and the residue was mixed with ethyl acetate (100 ml). The ethyl acetate layer was washed with 1.0 N hydrochloride solution (100 ml), brine (100 ml), dried over magnesium sulfate, and evaporated under reduced pressure to give a crude product, which was purified by silica gel column with hexane/ethyl acetate as eluent to give 2.14 g of pale-yellow oil product. Molecular Formular: C₄₇H₇₂O₃; Exact Mass: 684.55; m/z: 685.55 (M+1)⁺, 707.54 (M+Na)⁺. ¹H NMR: (400 MHz, DMSO-d₆): 0.75-0.87 (m, 12H), 0.93 (d, 6H), 0.98-1.08 (m, 2H), 1.01 (s, 3H), 1.15-1.30 (m, 14H), 1.30-1.40 (m, 2H), 1.42-1.52 (m, 5H), 1.53-1.59 (m, 2H), 1.60 (m, 2H), 1.67 (s, 3H), 1.85 (m, 2H), 2.00 (s, 3H), 2.06 (s, 3H), 2.30 (s, 3H), 2.69 (m, 2H), 6.03 (s, 1H), 6.17 (m, 1H), 6.28 (m, 1H), 6.48 (d, 1H), 6.69 (d, 1H), 6.73 (d, 1H), 6.77 (d, 1H), 7.14 (dd, 1H).

### Example 3. Synthesis of 3-tert-butyl-4-hydroxyphenyl retinoate

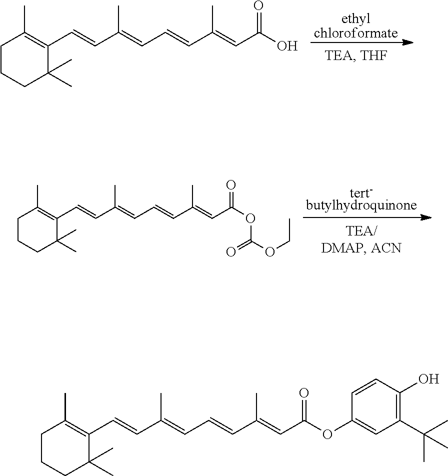 [SMILES: CC1=C(/C=C/C(C)=C/C=C/C(C)=C/C(=O)O)C(C)(C)CCC1 ; "ethylchloroformate" ; CCOC(=O)OC(=O)/C=C(C)/C=C/C=C(C)/C=C/C1=C(C)CCCC1(C)C ; "tert-butylhydroquinone" ; CC1=C(/C=C/C(C)=C/C=C/C(C)=C/C(=O)Oc2ccc(O)c(C(C)(C)C)c2)C(C)(C)CCC1]

Retinoic acid (2.00 g, 6.66 mmol) was dissolved in anhydrous tetrahydrofuran (50 ml). Triethylamine (2.0 ml) was added, and the mixture was stirred for five minutes. The solution of ethyl chloroformate (0.72 g, 6.66 mmol) in tetrahydrofuran (1 ml) was added dropwise at 0° C. The mixture was allowed to warm to room temperature and stirred for two hours.

Hexane (50 ml) was added and the triethylamine hydrochloride was collected by filtration. The filtrate was evaporated under reduced pressure.

The yellow residue was dissolved in anhydrous acetonitrile (50 ml), and tert-butylhydroquinone (TBHQ, 1.64 g, 9.99 mmol) was added. After the mixture was stirred for five minutes, triethylamine (5 ml) and 4-dimethylaminopyridine (1.00 g) were added. The mixture was warmed to 50° C. and stirred for four hours.

Most of solvent was evaporated under reduced pressure, and the residue was mixed with ethyl acetate (100 ml). The ethyl acetate layer was washed with 1.0 N hydrochloride solution (100 ml), brine (100 ml), dried over magnesium sulfate, and evaporated under reduced pressure to give a crude product, which was purified by silica gel column with hexane/ethyl acetate as eluent to give 1.89 g yellow crystal powder. Molecular Formular: C₃₀H₄₀O₃; Exact Mass: 448.30; m/z: 449.30 (M+1)⁺, 471.28 (M+Na)⁺. ¹H NMR: (400 MHz, DMSO-d₆): 1.01 (s, 6H), 1.32 (s, 9H), 1.43 (m, 2H), 1.57 (m, 2H), 1.69 (s, 3H), 1.99 (m, 2H), 2.00 (s, 3H), 2.33 (s, 3H), 6.03 (s, 1H), 6.20 (t, 1H), 6.28 (t, 2H), 6.49 (d, 1H), 6.75 (d, 2H), 6.80 (m, 1H), 7.13 (dd, 1H), 9.37 (s, 1H).

### Example 4. Analysis of Agonist Activity for RAR (Retinoic Acid Receptor)

### Methods

Reporter Cells (Indigo Biosciences, catalog #IB02201, IB02101, IB00821) were used in the assays. The reporter cells expressed a receptor hybrid in which the native N-terminal DNA binding domain (DBD) had been replaced with that of the yeast Gal4 DBD. The reporter gene, firefly luciferase, is functionally linked to the Gal4 upstream activation sequence (UAS). Receptor assays were performed as follows, Steps 1 to 3.

Step 1: A suspension of Reporter Cells was prepared in Cell Recovery Medium (CRM: containing charcoal-stripped FBS) and 100 μL of the Reporter Cell suspension was dispensed into each of the wells of a white 96-well assay plate. Step 2: Test and reference compound master stocks were prepared as solutions at 1,000× concentration in DMSO relative to the final treatment concentration. This intermediate stock was subsequently diluted directly into INDIGO Biosciences's Compound Screening Medium (CSM; containing charcoal-stripped FBS) to generate ‘2×-concentration’ treatment media. 100 μL of each prepared treatment medium was dispensed into duplicate assay wells pre-dispensed with a 100 μL suspension of Reporter Cells, thereby achieving the desired final treatment concentrations. The concentration of residual DMSO in all assay wells was 0.1% for the test and reference compounds. Assay plates were incubated at 37° C., 5% CO2 and ˜70% humidity for 23 hours. DMSO control was used to determine background activity. Retinoic acid was used with its EC100 concentration to show 100% activity of each receptor. Retinol served as positive control.

Step 3: Following the incubation period, treatment media were discarded and 100 μL/well of Luciferase Detection Reagent was added per well to determine receptor activity in terms of relative luminescence units (RLUs). Data processing: Fold activation: [Ave RLU Test Cmpd/Ave RLU DMSO].

### Results

TABLE 1
Agonist activities of (±)α-tocopheryl retinoate, (+)δ-tocopheroyl retinoate, and
3-tert-butyl-4-hydroxyphenyl retinoate to RAR alpha, beta and gamma receptor

| | RAR alpha | | RAR beta | | RAR gamma | |
|---|---|---|---|---|---|---|
| | Concen- | Fold | Concen- | Fold | Concen- | Fold |
| | tration | Activation | tration | Activation | tration | Activation |
| Control | 0.1% | 1 | 0.1% | 1 | 0.1% | 1 |
| DMSO | | | | | | |
| Retinoic acid | 2.5 μM | 4526 | 0.2 μM | 318 | 0.1 μM | 706 |
| Retinol | 0.01 μM | 1.77 | 0.01 μM | 1.63 | 0.01 μM | 2.28 |
| | 0.1 μM | 6.35 | 0.1 μM | 4.91 | 0.1 μM | 18.5 |
| | 1 μM | 518 | 1 μM | 127 | 1 μM | 368 |
| (±)α-tocopheryl | 0.01 μM | 1.27 | 0.01 μM | 0.882 | 0.01 μM | 1.79 |
| retinoate | 0.1 μM | 9.14 | 0.1 μM | 6.33 | 0.1 μM | 23.8 |
| | 1 μM | 552 | 1 μM | 149 | 1 μM | 365 |
| (+)δ- | 0.01 μM | 0.620 | 0.01 μM | 1.57 | 0.01 μM | 1.10 |
| Tocopheroyl | 0.1 μM | 5.25 | 0.1 μM | 3.96 | 0.1 μM | 8.45 |
| retinoate | 1 μM | 251 | 1 μM | 53.4 | 1 μM | 315 |
| 3-tert-Butyl-4- | 0.01 μM | 1,573 | 0.01 μM | 232 | 0.01 μM | 541 |
| hydroxyphenyl | 0.1 μM | 2,768 | 0.1 μM | 287 | 0.1 μM | 1,010 |
| retinoate | 1 μM | 3,414 | 1 μM | 331 | 1 μM | 701 |

As shown in Table 1, (±)α-tocopheryl retinoate, (+)δ-tocopheroyl retinoate, and 3-tert-butyl-4-hydroxyphenyl retinoate exhibited agonist activity towards RAR alpha, beta, and gamma receptors. In particular, 3-tert-butyl-4-hydroxyphenyl retinoate showed higher activity than retinol at all tested concentrations and exhibited a comparable activity to retinoic acid.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. An ester of Formula (I): 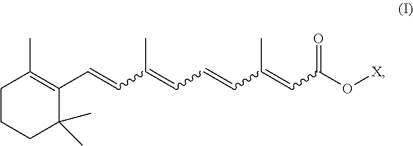
or a tautomer, isotopically labeled compound, solvate, polymorph, or co-crystal thereof, wherein X is of the formula:

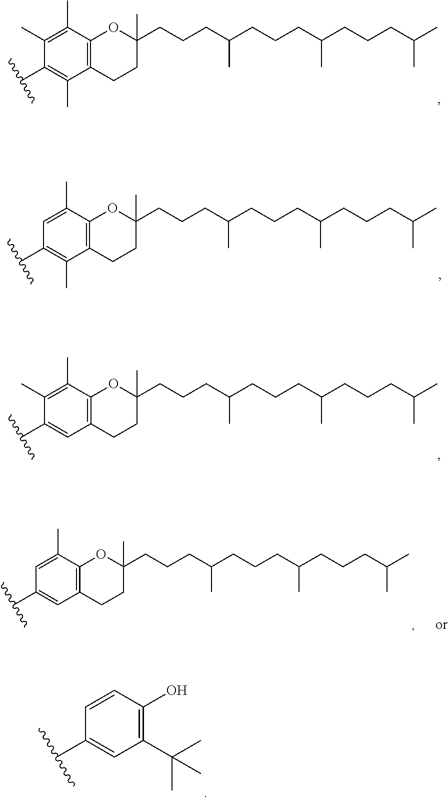

2. The ester of claim 1, wherein X is of the formula:

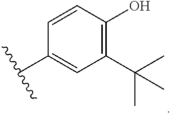 [SMILES: *c1ccc(O)c(C(C)(C)C)c1]

3. The ester of claim 1, wherein the ester is of the formula: 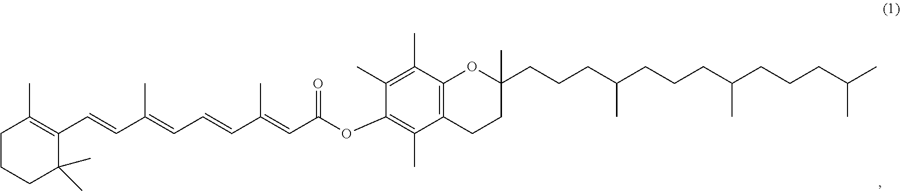 [SMILES: CC1=C(/C=C/C(C)=C/C=C/C(C)=C/C(=O)Oc2c(C)c(C)c3c(c2C)CCC(C)(CCCC(C)CCCC(C)CCCC(C)C)O3)C(C)(C)CCC1]
or a tautomer, isotopically labeled compound, solvate, polymorph, or co-crystal thereof.

4. The ester of claim 1, wherein the ester is of the formula: 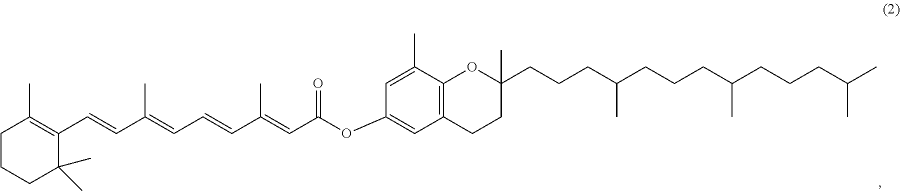 [SMILES: CC1=C(/C=C/C(C)=C/C=C/C(C)=C/C(=O)Oc2cc(C)c3c(c2)CCC(C)(CCCC(C)CCCC(C)CCCC(C)C)O3)C(C)(C)CCC1]
or a tautomer, isotopically labeled compound, solvate, polymorph, or co-crystal thereof.

5. The ester of claim 1, wherein the ester is of the formula: 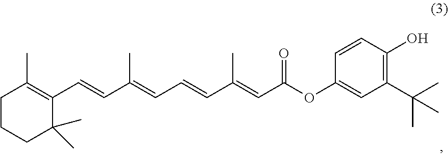 [SMILES: CC1=C(/C=C/C(C)=C/C=C/C(C)=C/C(=O)Oc2ccc(O)c(C(C)(C)C)c2)C(C)(C)CCC1]
or a tautomer, isotopically labeled compound, solvate, polymorph, or co-crystal thereof.

6. The ester of claim 1, wherein the ester is of the formula: 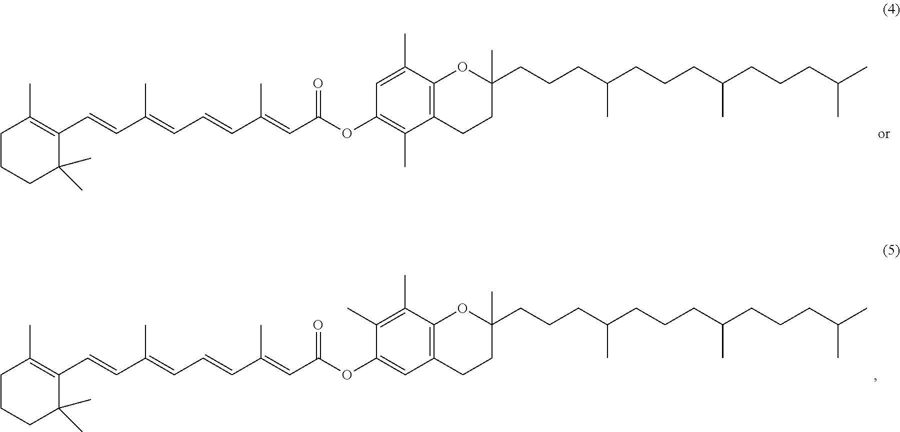 [SMILES: CC1=C(/C=C/C(C)=C/C=C/C(C)=C/C(=O)Oc2cc(C)c3c(c2C)CCC(C)(CCCC(C)CCCC(C)CCCC(C)C)O3)C(C)(C)CCC1 ; CC1=C(/C=C/C(C)=C/C=C/C(C)=C/C(=O)Oc2cc3c(c(C)c2C)OC(C)(CCCC(C)CCCC(C)CCCC(C)C)CC3)C(C)(C)CCC1]
or a tautomer, isotopically labeled compound, solvate, polymorph, or co-crystal thereof.

7. The ester of claim 1, or a tautomer or isotopically labeled compound thereof.

8. A method of producing the ester of claim 1, or a tautomer, isotopically labeled compound, solvate, polymorph, or co-crystal thereof, the method comprising incubating a second reaction mixture for a second time duration sufficient to produce the ester, tautomer, isotopically labeled compound, solvate, polymorph, or co-crystal, wherein the second reaction mixture comprises:
(a) an anhydride of Formula (B): 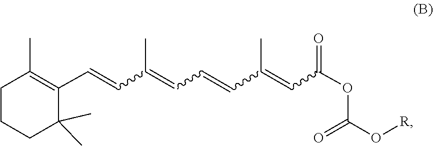
or a tautomer, isotopically labeled compound, solvate, polymorph, or co-crystal thereof, wherein R is substituted or unsubstituted alkyl;
(b) an alcohol of Formula (C):
HO—X (C),
or a tautomer, isotopically labeled compound, solvate, polymorph, or co-crystal thereof;
(c) a second solvent;
(d) optionally a second base; and
(e) optionally an esterification catalyst.

9. The method of claim 8, wherein Formula (B) is of the formula:

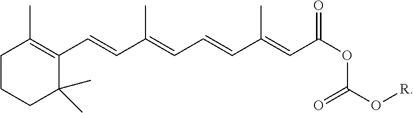

10-23. (canceled)

24. The method of claim 8, wherein the method further comprises incubating a third reaction mixture comprising the second reaction mixture and ethanolamine, or a salt thereof, for a third time duration.

25-29. (canceled)

30. The method of claim 8, wherein the method further comprises purifying the ester, or a tautomer, isotopically labeled compound, solvate, polymorph, or co-crystal thereof.

31. (canceled)

32. The method of claim 8, wherein the method further comprises incubating a first reaction mixture for a first time duration sufficient to produce (a) of the second reaction mixture, wherein:
the first reaction mixture comprises:
(a) an acid of Formula (A1): 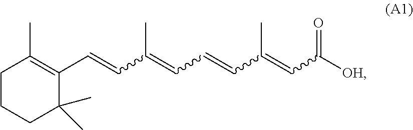 [SMILES: CC(C=CC1=C(C)CCCC1(C)C)=CC=CC(C)=CC(=O)O]
or a tautomer, isotopically labeled compound, salt, solvate, polymorph, or co-crystal thereof;
(b) a chloroformate of Formula (A2):
Cl—C(═O)—O—R (A2),
or an isotopically labeled compound, solvate, polymorph, or co-crystal thereof, wherein R is substituted or unsubstituted alkyl;
(c) a first solvent; and
(d) optionally a first base; and
the step of incubating a first reaction mixture is prior to the step of incubating the second reaction mixture.

33-48. (canceled)

49. The method of claim 32, wherein the method further comprises purifying (a) of the second reaction mixture, wherein the step of purifying (a) of the second reaction mixture is prior to the step of incubating the second reaction mixture.

50-52. (canceled)

53. A composition comprising:
the ester of claim 1, or a tautomer, isotopically labeled compound, solvate, polymorph, or co-crystal thereof; and
optionally an excipient.

54-57. (canceled)

58. A composition comprising the ester, or a tautomer, isotopically labeled compound, solvate, polymorph, or co-crystal thereof, produced by the method of claim 8.

59. A kit comprising:
the ester of claim 1, or a tautomer, isotopically labeled compound, solvate, polymorph, or co-crystal thereof; and
optionally instructions for using the ester, tautomer, isotopically labeled compound, solvate, polymorph, or co-crystal.

60. A method of treating a skin disease in a subject in need thereof comprising administering to the subject in need thereof an effective amount of the ester of claim 1, or a tautomer, isotopically labeled compound, solvate, polymorph, or co-crystal thereof.

61. A method of preventing a skin disease in a subject in need thereof comprising administering to the subject in need thereof an effective amount of the ester of claim 1, or a tautomer, isotopically labeled compound, solvate, polymorph, or co-crystal thereof.

62-68. (canceled)

69. A method of slowing the ageing of the skin or improving the appearance of the skin of a subject comprising administering to the subject an effective amount of the ester of claim 1, or a tautomer, isotopically labeled compound, solvate, polymorph, or co-crystal thereof.

70. (canceled)

71. A method of modulating a retinoid receptor in a subject, biological sample, tissue, or cell comprising administering to the subject or contacting the biological sample, tissue, or cell with an effective amount of the ester of claim 1, or a tautomer, isotopically labeled compound, solvate, polymorph, or co-crystal thereof.

72-75. (canceled)
